(19) United States
(12) Patent Application Publication
(10) Pub. No.: US 2025/0288309 A1
(43) Pub. Date: Sep. 18, 2025

(54) INTRAVASCULAR LITHOPLASTY SYSTEM WITH IMPROVED DURABILITY, EFFICIENCY AND PRESSURE OUTPUT VARIABILITY

(71) Applicant: Cardiovascular Systems, Inc., Santa Clara, CA (US)

(72) Inventors: Tim HIDANI, Mound, MN (US); Alexander P. THOME, Minnetonka, MN (US); Daniel SCHMITT, Belle Plaine, MN (US); Kevin CORNELIUS, Wayzata, MN (US); Eric KASEN, Bismarck, ND (US)

(21) Appl. No.: 19/225,508

(22) Filed: Jun. 2, 2025

Related U.S. Application Data
(63) Continuation of application No. PCT/US2023/085868, filed on Dec. 23, 2023.
(60) Provisional application No. 63/477,007, filed on Dec. 23, 2022.

(51) Int. Cl. A61B 17/22; A61B 17/00

(52) U.S. Cl. CPC A61B 17/22022; A61B 2017/00154; A61B 2017/00477; A61B 2017/00557; A61B 2017/00929; A61B 2017/22065

(57) ABSTRACT

Various embodiments of the systems, methods and devices are provided for breaking up calcified lesions in an anatomical conduit wherein exemplary systems generate an electrical arc between two spaced-apart electrodes disposed within a fluid-filled balloon.

### CROSS-REFERENCE TO RELATED APPLICATIONS

This application is a continuation of International application number PCT/US2023/085868, filed Dec. 23, 2023, and entitled Intravascular Lithoplasty System With Improved Durability, Efficiency and Pressure Output Variability, which claims priority to provisional application No. 63/477,007, filed Dec. 23, 2022, and entitled INTRAVASCULAR LITHOPLASTY AND/OR ANGIOPLASTY BALLOON SYSTEM WITH IMPROVED BALLOON MOUNT AND ELECTRODES, the entire contents of which applications are incorporated herein by reference as fully as if reproduced herein in full, for all purposes.

### STATEMENT REGARDING FEDERALLY SPONSORED RESEARCH OR DEVELOPMENT

None

### BACKGROUND OF THE INVENTION

### Field of the Invention

The invention relates to systems, devices and methods for breaking up calcified lesions in an anatomical conduit. In one aspect, an electrical arc is generated between two spaced-apart electrodes disposed within a fluid-filled member, creating pressure waves. In another aspect, a fluid-filled member may be inflated and deflated to open occluded blood vessels, including but not limited to calcified occlusions.

### Description of the Related Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with an inner diameter that is similar to the native vessel in the occlusion area and, thereby, improving blood flow. Generally, known IVL devices include a voltage pulse generator in operative communication with one or more pairs of electrodes mounted on a catheter and within an inflatable balloon.

Intravascular lithotripsy systems, devices and methods have been described by Applicant. See PCT/US2022/074607, filed Aug. 5, 2022 and entitled “INTRAVASCULAR LITHOTRIPSY BALLOON SYSTEMS, DEVICES AND METHODS”, the entire contents of which are hereby incorporated by reference.

As illustrated in FIG. 1, a diagrammatic layout of portions of an exemplary IVL system 12 is provided. The illustrative IVL system 12 comprises a catheter assembly 114 including an elongate body, embodied as a catheter having guidewire 15, and a fluid-filled member 16 configured to contain conductive fluid therein, exemplified by an inflatable balloon, disposed near one end of the body and arranged to receive fluid for inflation to facilitate IVL therapy. A set of dischargeable spaced-apart electrodes 18 are shown arranged within the exemplary balloon 16, at least some of which are spaced apart by a gap 17 from each other to create a spark or electrical arc between the spaced-apart electrodes 18.

The IVL system embodiments described herein may be used in connection with electrodes that are within a fluid-filled member 16 configured to contain a fluid, e.g., a conductive fluid, therein. The fluid-filled member 16 embodiments may include an inflatable balloon as shown in FIG. 1, which may be compliant or non-compliant and serves to contain the fluid such that the spaced-apart electrodes 18 are submerged within the contained fluid. In addition, the fluid-filled member 16 may comprise a fillable member that is at least partially rigid and/or not flexible. In other embodiments, the fluid-filled member 16 may contain the fluid therein and wherein the spaced-apart electrodes 18 are located or submerged within the contained fluid.

Alternatively, the IVL system control embodiments of the present disclosure may be used in connection with electrodes that are not located or surrounded by a fluid-filled or fillable member 16. In these embodiments, the IVL system may comprise spaced-apart electrodes 18 that may be continuously or periodically exposed to saline or other fluid and, during the exposure, the IVL system may generate an electrical arc between the spaced-apart electrodes 18.

The spaced-apart electrodes 18 are arranged in communication (as suggested by dashed line conductors) with an electric pulse generation system 20 to receive high voltage electrical energy for spark generation to create pressure waves for IVL therapy. In the illustrative embodiment, one electrode may be grounded and the other provided with high voltage from the electric pulse generation system 20, although in some embodiments, any voltage differential may be applied. The electric pulse generation system 20 includes an IVL control system 22 comprising a processor 24 configured for executing instructions stored on memory 26 and communications signals via circuitry 28 for IVL operations according to the processor governance. The processor 24, memory 26, and circuitry 28 are arranged in communication with each other (as suggested via dashed lines) to facilitate disclosed operations.

Appropriate control of such high-energy systems can also require achieving sufficient energy at the discharge site. Given the high-energy environment and microscale time periods for electronic discharge, desirable energy control within such IVL devices and systems can be challenging. Moreover, adaptable control methodologies may offer advantages to IVL effectiveness. Adjustable energy delivery can increase efficient power application, which can reduce risk to the patient. For example, beginning with a predetermined starting voltage threshold and defining a predetermined upper voltage threshold to form an acceptable voltage window may be proved. The acceptable voltage window may be coupled with series of generated voltage pulses of magnitudes that are confirmed to be within the acceptable voltage window. If, e.g., the magnitudes of the series of generated voltage pulses are below the predetermined upper voltage threshold, then the target voltage may be increased by a predetermined amount and another series of generated voltage pulses is executed. The embodiments of the IVL systems, devices and methods within the present disclosure includes operation for adjusting the total electrical energy provided to the set of electrodes for a given pulse. Such control systems for intravascular lithotripsy systems, devices and methods have been described by Applicant. See PCT/US2023/79209, filed Nov. 9, 2023 and entitled “CONTROL OF IVL SYSTEMS, DEVICES AND METHODS THEREOF”, the entire contents of which are hereby incorporated by reference.

A portion of an exemplary known competitive IVL device is shown in cross-section in FIG. 2, viewed in cross-section along a line cutting through an IVL balloon which surrounds, inter alia, a catheter body.

Thus, the prior art inflation port is in fluid communication with the fill lumen/passageway. The fill lumen/passageway of the competitive IVL device of FIG. 2 is formed between the inner surface of the catheter body and an outer surface of a sleeve that encompasses wire conductors (terminating at electrodes that are located along the catheter and within the balloon). Thus, the wire conductors of the known device are not exposed to the fluid within the fill lumen/passageway. Electrodes that are located within the balloon and electrically connected with the wire conductors are not covered by the encompassing sleeve and, therefore, are exposed to fluid within the balloon. In addition, the known competitive device comprises an angioplasty balloon that is adhered at both the proximal end of the balloon and the distal end of the balloon to an outer surface of the catheter body, with the fill lumen/passageway into the balloon's interior being formed within the catheter body and defined by a space between the sleeve and the inner surface of the catheter body. Accordingly, the competitive known IVL balloon is adhered at its proximal end and its distal end to one structure, that is, the outer surface of the catheter body.

In addition, the competitive known IVL device of FIG. 2 comprises the catheter body, and the guidewire member (which defines the guidewire lumen) both extending through the balloon. Both of these structures continue distally beyond a distal end of the balloon to a distal tip. As a result, the distal tip of the known competitive device comprises a stiffness and deformability that can be improved upon to, in tum, improve the device's ability to translate tortuous vasculature and reduce the potential of damaging the vasculature during translation.

Further, the sleeve shown in FIG. 2 adds a layer of material that adds crossing profile thickness, increases complexity and takes up a portion of the area of the fill lumen/passageway, thereby reducing the available volume fill lumen/passageway during inflation and/or deflation cycles.

FIG. 3 graphically illustrates the drop in impedance of a current leader across a spark gap defined between two spaced-apart electrodes in an IVL system such as shown in FIG. 1 following application of voltage to one of two spaced-apart electrodes as the current leader develops into an electrical arc between the spaced-apart electrodes. This, in tum, causes the power dissipated in the electrical arc to peak sharply while the voltage and current between the electrodes are both relatively high. The current reaches a peak and the voltage drops, both very rapidly, indicating that an electrical arc between the spaced-apart electrodes is present, or has occurred. The peak of the power dissipated in the electrical arc indicates the relatively short time interval during which all the useful work of heating the growing leader into an arc is performed. The graphic illustration of FIG. 3 is exemplary of one aspect of an IVL procedure that produces pressure waves.

It would be advantageous to provide an IVL system and/or device that reduces the risk of trauma during translation of the angioplasty balloon to an anatomical location of interest and that reduces the complexity of construction of a lithotripsy system.

Moreover, it would be advantageous to provide an IVL system and/or device that comprises a reduced crossing profile, a more flexible tip and a kink-resistant shaft.

Known IVL devices also comprise a spark gap between electrode pairs that, when a sufficiently high voltage is applied to a first electrode, facilitates a spark or electrical arc of current from the first electrode across the spark gap to a second electrode in the electrode pair. This process results in loss of material, or erosion, from each of the electrodes in the electrode pair. Known spark gaps are generally arranged axially, i.e., with terminal faces of wire conductors spaced apart from each other and facing each other in an axial spark gap configuration. In this case, the electrical arc involves the terminal or distal face of at least one electrode in the electrode pair. Continued arcing across the spark gap during an IVL procedure with these known devices results in erosion of material from each electrode involved in the electrical arc. Each of these arcs causes the spark gap to slightly increase in size and/or short, with the full procedural set of arcs resulting in an appreciable increase in spark gap size which may lead to unpredictability in generating an electrical arc. Alternatively, 1st and 2nd electrodes may be arranged concentrically, with the spark gap defined in a radial direction, wherein the 1st and 2nd electrodes are not arranged along a common radial or circumferential plane. As will be demonstrated herein, this arrangement generates pressure outputs with relatively high variability. In addition, known IVL devices produce a resultant pressure output, or “shockwave” or “pressure wave” that over a series of electrical arcs, decreases in magnitude as the IVL procedure is executed. Moreover, the highly variable pressure outputs produced by the known IVL devices can lead to unpredictable, or undesirable, outcomes and perhaps contributes to balloon instability over time due to the variability of the pressure outputs placing stress on the balloon material. Further, known IVL coronary devices are configured to produce a total maximum number of 120 pulses per catheter at a frequency of 1 Hz. Known IVL peripheral device are configured to produce a total maximum number of 300 pulses per catheter at 1 Hz.

It would be advantageous to provide an IVL device or system that is designed to maintain a desired spark gap distance between spaced-apart electrodes across the execution of a full IVL procedure and produce tightly controlled output pressures having a much tighter data spread from high to low data points, and lower standard deviation as compared to the known IVL devices.

It would also be desirable to provide an IVL device or system that is more durable and more efficient than known devices through structural and operational improvements to provide an IVL device or system that can provide up to, and more than, 300 voltage pulses per catheter in some embodiments and in other embodiments up to 500 voltage pulses per catheter, with a frequency of 1 to 5 Hz, with 2 Hz being a preferred frequency.

It would be advantageous to provide an IVL device or system with a catheter comprising key features that allow for improved pushability and kink resistance, particularly in the region of the balloon, as well as a reduced crossing profile in at least that region.

Various embodiments of the present invention address the advantageous, among others, discussed above.

### BRIEF DESCRIPTION OF THE SEVERAL VIEWS OF THE DRAWINGS

These drawings are exemplary illustrations of certain embodiments and, as such, are not intended to limit the disclosure

FIG. 1 illustrates a schematic view of an exemplary IVL device.

FIG. 2 illustrates a cutaway view of a known IVL device.

FIG. 3 illustrates a graphic illustration of a typical timing of applied voltage and current during application of voltage and generation of an electrical arc between spaced-apart electrodes.

FIG. 4 illustrates one embodiment of the present disclosure.

FIG. 5 illustrates one embodiment of the present disclosure.

FIG. 6 illustrates a side, cutaway view of a distal portion of an exemplary embodiment of the present disclosure.

FIG. 7A illustrates a side, cutaway view of an exemplary embodiment of the present disclosure.

FIG. 7B illustrates a side, cutaway view of an exemplary embodiment of the present disclosure.

FIG. 8 illustrates a side, cutaway view of a portion of the distal region of an exemplary embodiment of the present disclosure.

FIG. 9 illustrates a partial cutaway view of a portion of an exemplary embodiment of the present disclosure.

FIG. 10 illustrates a side, cutaway view of a portion of FIG. 6.

FIG. 11 illustrates a side, cutaway view of the embodiment of FIG. 10.

FIG. 12A illustrates an embodiment of a portion of a system of the present disclosure.

FIG. 12B illustrates an embodiment of a portion of a system of the present disclosure.

FIG. 12C illustrates an embodiment of a portion of a system of the present disclosure.

FIG. 12D illustrates an embodiment of a portion of a system of the present disclosure.

FIG. 13A illustrates an embodiment of a portion of a system of the present disclosure.

FIG. 13B illustrates an embodiment of a portion of a system of the present disclosure.

FIG. 14A illustrates a side, cutaway view of one embodiment of the present disclosure.

FIG. 14B illustrates a portion of device illustrated in FIG. 14A.

FIG. 15A illustrates one embodiment of the present disclosure.

FIG. 15B illustrates one embodiment of the present disclosure.

FIG. 15C illustrates one embodiment of the present disclosure.

FIG. 15D illustrates one embodiment of the present disclosure.

FIG. 15E illustrates a cross-sectional view of one embodiment of the present disclosure.

FIG. 16A illustrates one embodiment of the present disclosure.

FIG. 16B illustrates one embodiment of the present disclosure.

FIG. 16C illustrates one embodiment of the present disclosure.

FIG. 16D illustrates one embodiment of the present disclosure.

FIG. 17A illustrates one embodiment of the present disclosure.

FIG. 17B illustrates one embodiment of the present disclosure.

FIG. 17C illustrates one embodiment of the present disclosure.

FIG. 17D illustrates one embodiment of the present disclosure.

FIG. 18A illustrates one embodiment of the present disclosure.

FIG. 18B illustrates one embodiment of the present disclosure.

FIG. 19A illustrates one embodiment of the present disclosure.

FIG. 19B illustrates one embodiment of the present disclosure.

FIG. 20 illustrates a side view of one embodiment of the present disclosure.

FIG. 21 illustrates a schematic diagram of one embodiment of the present disclosure.

FIG. 22 illustrates a top cutaway view of one embodiment of the present disclosure.

FIG. 23 illustrates a block diagram of one embodiment of the present disclosure.

FIG. 24 illustrates a pressure plot comparing test and known IVL devices.

FIG. 25 illustrates a force tracking plot comparing tracking force through a tracking fixture for TEST and KNOWN IVL catheters.

### DETAILED DESCRIPTION OF THE INVENTION

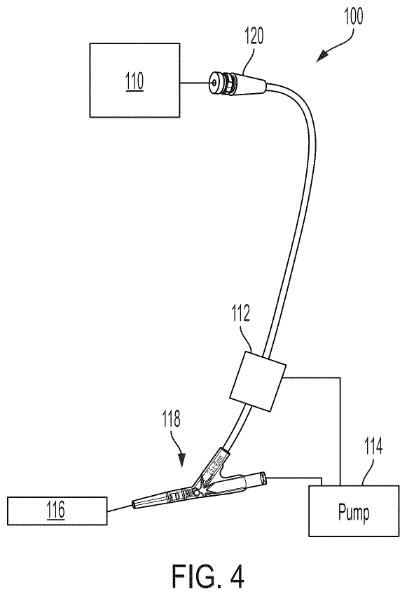

FIG. 4 illustrates an embodiment of an IVL system 100 of the present disclosure. A voltage pulse generator 110 is provided in operative connection and communication with a controller 112 configured to provide programmed operative instructions to the voltage pulse generator 110 and to a fluid reservoir/fluid pump device 114. The controller 112 and voltage pulse generator 110 are in operative electrical communication with wire conductors and pairs of electrodes as discussed above, wherein the wire conductors are disposed along the catheter's length and wherein the electrode pairs are disposed within the interior of an inflatable balloon that is positioned at or near a distal end of the catheter structure. The catheter and balloon structure are represented schematically by element number 116. A hub 118 allows operative connection and communication with the controller 112 and fluid reservoir/pump 114 and voltage pulse generator 110. In addition, connector 120 is in operative connection and communication with the controller 112 and provides operative electrical connection and communication with the wire conductors and electrode pairs. The controller 112 may comprise a processor for executing programmed instructions, for example initiating voltage pulses at a predetermined magnitude and frequency and in a predetermined pattern of pulses and magnitudes. The processor may be in operative communication and connection with a memory and a display. In some embodiments, the hub 118 may allow for over-the-wire guidewire access through a lumen defined within the catheter. In a preferred embodiment, a rapid exchange (RX) access is provided. Some embodiments of the controller 112 may comprise an EPROM comprising programmed instructions, for example and without limitation, initiating voltage pulses at a predetermined magnitude and frequency and in a predetermined pattern of pulses and magnitudes.

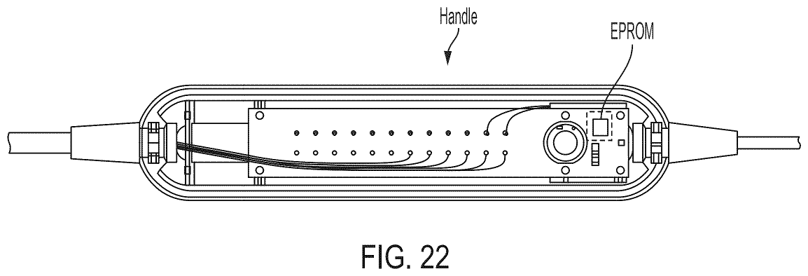

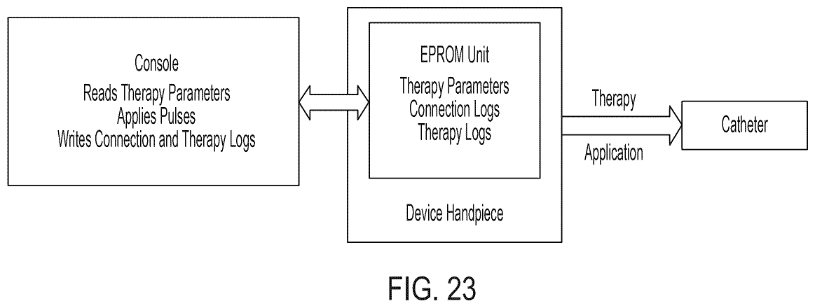

As discussed in FIGS. 22 and 23, some embodiments may comprise a handle may comprise an EPROM comprising programmed instructions, for example and without limitation, initiating voltage pulses at a predetermined magnitude and frequency and in a predetermined pattern of pulses and magnitudes. The EPROM may be in operative communication and connection with a console comprising a processor for executing programmed instructions, a memory in operative communication with the processor and, in some embodiments, the EPROM, and a display. In some embodiments, the hub 118 may allow for over-the-wire guidewire access through a lumen defined within the catheter.

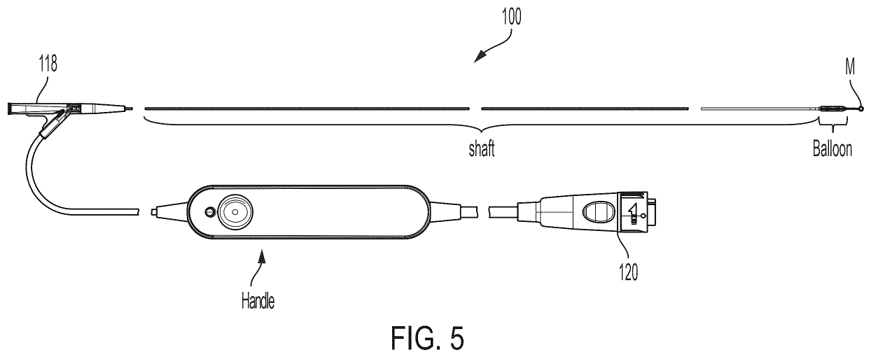

FIG. 5 shows a broken side view of the IVL system 100 comprising the a handle with a connector 120 configured to connect with the voltage pulse generator 110 of FIG. 4 and a console as described above. A removable mandrel M is shown inserted through a guidewire lumen defined along a portion of the catheter shaft, extending proximally through a flexible distal tip and through the defined guidewire lumen of the catheter.

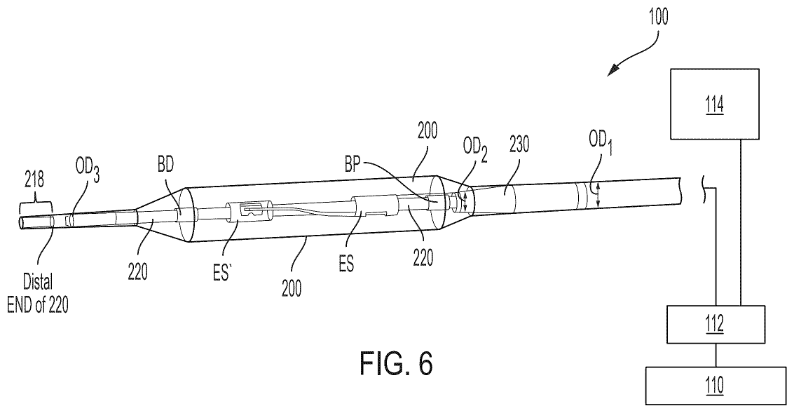

Distal portion embodiments of the catheter and balloon element 116 of the exemplary IVL system 100 of FIGS. 4 and 5 are illustrated in FIGS. 6-11.

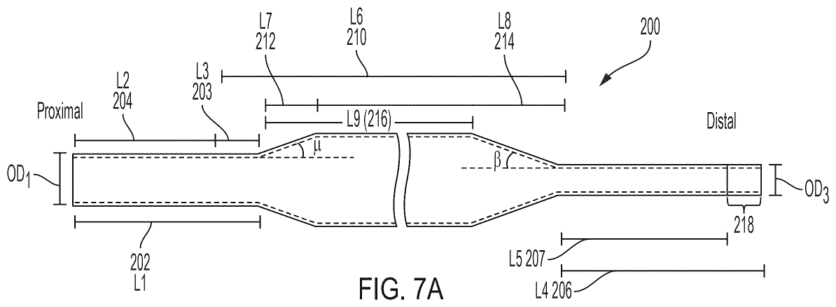

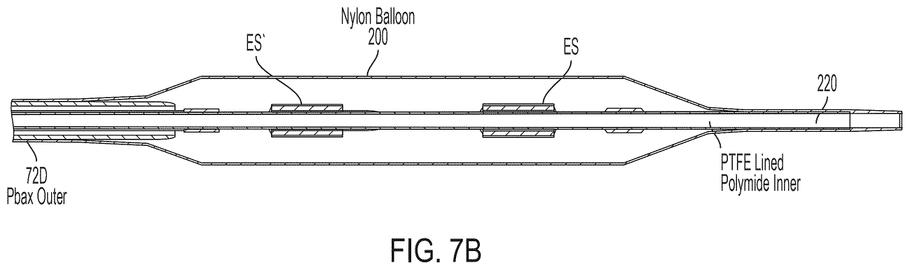

As best seen in FIG. 7A, an inflatable balloon 200 is provided and comprises a cylindrical proximal section 202, a cylindrical distal section 206 and an inflatable portion 210 comprising an unbonded section 203 of the cylindrical distal section 206, a tapering proximal section 212, a tapering distal section 214 and a substantially cylindrical section 216 disposed between the tapering proximal and distal sections 212,214.

A proximal portion 207 of the cylindrical distal section 206 of the balloon 200 surrounds, and is bonded or sealed in a watertight engagement against an outer surface of an elongate member 220 as best shown in FIG. 6. A distal region of the cylindrical distal section 206 extends, e.g., is extruded, beyond a distal end of the elongate member 220 to form an atraumatic, flexible tip 218.

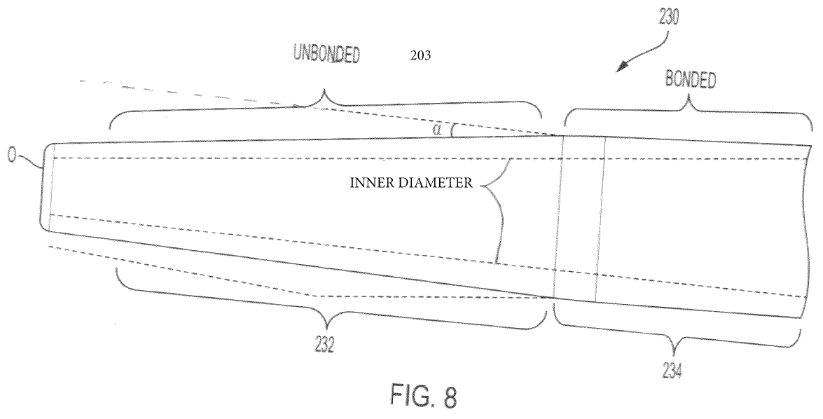

A proximal portion 204 of the cylindrical proximal section 202 of the balloon surrounds, and is sealed or bonded in a watertight engagement against, the non-tapering outer surface 234 of the tapering outer member 230 (see FIG. 8), and is not sealed or bonded against any portion of the elongate member 220 which is received within, and extends distally from, a distal end of the tapering outer member 230. A distal length comprising an unbonded section 203 of the cylindrical proximal section 202 of the balloon 200 surrounds the distal tapering section 232 of the tapering outer member 230, but is not bonded or sealed against any portion of the tapering outer member 230. As a result, the inflatable section of the balloon 200 comprises: the unbonded section 203 of the cylindrical proximal section 202, the proximal tapering section 212, the distal tapering section 214 and the substantially cylindrical section 216 disposed between the proximal and distal tapering sections 212, 214.

The tapering outer member 230 comprises an outer diameter that is larger than an outer diameter of the elongate member 220, and is configured to receive the elongate member 220.

In addition, a length of a fluid conveying pipe P is defined along the length of the device and between the concentric arrangement of the outer surface of the elongate member 220 and the inner surface of the tapering outer member 230. The remaining portion of the fluid conveying pipe P is defined proximally along the catheter shaft. The fluid conveying pipe P is provided the exclusive path for fluid communication between the fluid reservoir/pump 114 and the inflatable section 210 of the balloon 200. The fluid conveying pipe P terminates distally relative to the distal end of the tapering outer member 230 where an opening O is defined for fluid flow into, and out of, the balloon 200.

It is significant to note that the distal end of the tapering outer member 230, and therefore the opening O for fluid flow, extends distally beyond a distal end of the proximal cylindrical section 202 of the balloon. As a result, the proximal cylindrical section 202 of the balloon surrounds at least a portion of the proximal non-tapering, or cylindrical section, 234 of the tapering outer member 230, and is sealed or bonded against the non-tapering or cylindrical (proximal) section's 234 outer surface in a watertight seal. A distal end region of the tapering outer member 230 is, however, not surrounded by the proximal cylindrical section 202 of the balloon. Instead, the distal end region of the tapering outer member 230, and its opening O, extends into the proximal tapering section 212 of the balloon 200.

As a result of the watertight sealing mechanism described above, and the location of the opening O for fluid flow into and out of the inflatable portion 210 of the balloon 200, the proximal cylindrical section 202 of the balloon 200 plays no role in the movement of fluid into or out of the inflatable portion 210 of the balloon 200. Similarly, the inflatable portion 210 of the balloon 200 also plays no role in the inflation/deflation or degassing of the fluid, or the movement of fluid into or out of the inflatable portion 210 of the balloon 200. The inflatable portion 210 simply receives the incoming fluid from, or delivers outgoing fluid into, the opening O at the distal end of the tapering outer member 230.

FIG. 8 shows a distal portion of the tapering outer member 230, including a tapering section 232 comprising a tapering angle a. The tapering section 232 in this embodiment comprises the unbonded section 203 of the balloon 200, wherein the proximal cylindrical section 203 of balloon 200 (see FIG. 7A) surrounds a portion of the tapering section 232, but is not adhered or bonded or sealed against the outer surface of the tapering section 232. Proximal of the tapering section 232 the tapering outer member 230 comprises a cylindrical section 234 of substantially constant diameter. This is indicated as the bonded section and provides an outer surface which the proximal portion 204 of the proximal cylindrical section 202 of the balloon 200 surrounds and is sealed or bonded in a watertight engagement. Sides of the fluid conveying pipe P is discussed above comprise a resulting inner diameter that is shown in dashed lines, wherein the pipe P terminates at an opening O at the distal end of the tapering outer member 230. As shown by the inner set of dashed lines, the inner diameter tapers distally within the tapering section 232. In other embodiments, the pipe P may be of substantially constant diameter through both the cylindrical section 234 and the tapering section 232, wherein the wall thickness of the pipe may neck down, or be made thinner moving in the distal direction at tapering section 232 while retaining a constant inner diameter through the cylindrical section 234 and the tapering section 232. In other embodiments, as in FIG. 8, the contours of the pipe P may taper downward with the tapering angle a of the tapering section 232. In some embodiments the inner diameter of the pipe P may comprise a constant diameter.

As shown in FIGS. 7A and 7B, and with continued reference to FIGS. 6, 8 10 and 11, the cylindrical proximal section 202 of the balloon 200 comprises a length L1 and an outer diameter OD1. Even though the length L1 of the cylindrical proximal section 202 surrounds the tapering outer member 230, only a proximal portion 204 of the cylindrical proximal section 202 is bonded or sealed in a watertight configuration against an outer surface of the elongate member. The proximal portion 204, also referred to as the proximal watertight sealed or bonded portion 204 has a length L2 which is less than L1. Finally, the cylindrical proximal section 202 further comprises, on its distal end, an unbonded section 203 having a length of L3 and that surrounds the tapering outer member 230, but is not bonded or sealed to the elongate member, wherein length L3 is less than L2 and L1, such that L2 plus L3 equals L1.

The balloon 200 further comprises a cylindrical distal section 206 having an overall length of L4, including the distal tip 218. Without including the distal tip 218, the proximal portion 207 of the cylindrical distal section 206 that surrounds, and is sealed or bonded in a watertight engagement against the outer surface of, the elongate member 220 comprises a length of LS, which is shorter than L4. Accordingly, the distal tip 218 extends distally beyond the distal end of the elongate member 220 for a distance to provide an atraumatic tip and to facilitate translation through the vasculature.

As described above, the inflatable portion 210 of the balloon 200 comprises the unbonded section (length L3) 203, the tapering proximal section 212 and the tapering distal section 214, and the substantially cylindrical section 216 disposed therebetween. Accordingly, the length of the inflatable section is L6 which includes the unbonded section 203 of the proximal cylindrical section of length L3, the proximal tapering section 212 of length L7, the distal tapering section 214 of length L8, and the substantially cylindrical section 216 of length L9, for an overall inflatable section length of L6.

The tapering outer member 230 may taper down to a smallest outer diameter OD2 at its distal end that is smaller than outer diameter OD1 which is the effective outer diameter of the proximal cylindrical section 202 as well as the outer diameter of the non-tapering portion 234 of the tapering outer member 230. In this embodiment, an inner diameter of the balloon's cylindrical proximal section 202 may be substantially equivalent to the outer diameter of the tapering outer member at OD1, which is the non-tapering portion 234 of the tapering outer member to which the proximal cylindrical section is watertight bonded or sealed. Similarly, an inner diameter of the proximal portion 207 of the balloon's cylindrical distal section 206 that is watertight bonded or sealed to the elongate member 220 may be substantially equivalent to the outer diameter of the elongate member 220 at OD3.

As can be seen in FIGS. 6 and 7A, the outer diameter of the outer member to which the balloon's cylindrical proximal section is sealed or bonded, that is OD1, may be greater than the outer diameter of the elongate member with outer diameter OD3.

As noted, the elongate member 220 is received within tapering outer member 230. As a result, the balloon 200 is sealed against two distinct structures. On the proximal side, the balloon 200 is sealed against the non-tapering outer surface 234 of the outer member 230 while on the distal side, the balloon 200 is sealed against the outer surface of the elongate member 220.

The balloon's distal tip 218 is preferably flexible and comprises a conduit defined therethrough and that aligns with a conduit defined through the elongate member to allow for, inter alia, guidewire access.

In some embodiments, the larger proximal side outer diameter OD1 as compared with smaller distal side outer diameter OD3, may produce a tapering angle μ for the tapered proximal section of the balloon that is different from, e.g., smaller than, the tapering angle for the tapered distal section of the balloon. These tapering angles are measured with reference to the dashed lines of FIG. 7A, which are collinear with the non-tapering outer surface of the outer member (tapering angle μ, and the outer surface of the elongate member (tapering angle). In some embodiments, the length L7 of the tapered proximal section may be shorter than the length LS of the tapered distal section.

Because of these exemplary relative dimensions, as shown in both FIGS. 7A and 7B, embodiments of the balloon 200 in the inflatable section 210 may be longitudinally asymmetric. In particular, the inflatable section 210 may be longitudinally asymmetric with a smaller tapering angle μ at the proximal tapering section 212 than the tapering angle at the distal tapering section 214 of the balloon 200, which may help facilitate access into tight lesions. In other embodiments, the tapering angles μ and may be substantially the same. In addition, embodiments of the distal cylindrical section 206 of the balloon 200 comprise a smaller outer diameter at OD3 than the proximal cylindrical section's 202 outer diameter, which may also facilitate access into tight lesions. Stated differently, the crossing profile of the device distal of the substantially cylindrical section 216 of the balloon 200 is smaller than the crossing profile of the device proximal of the substantially cylindrical section 216 of the balloon 200.

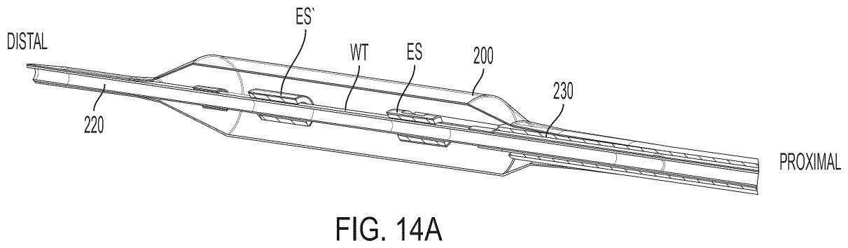

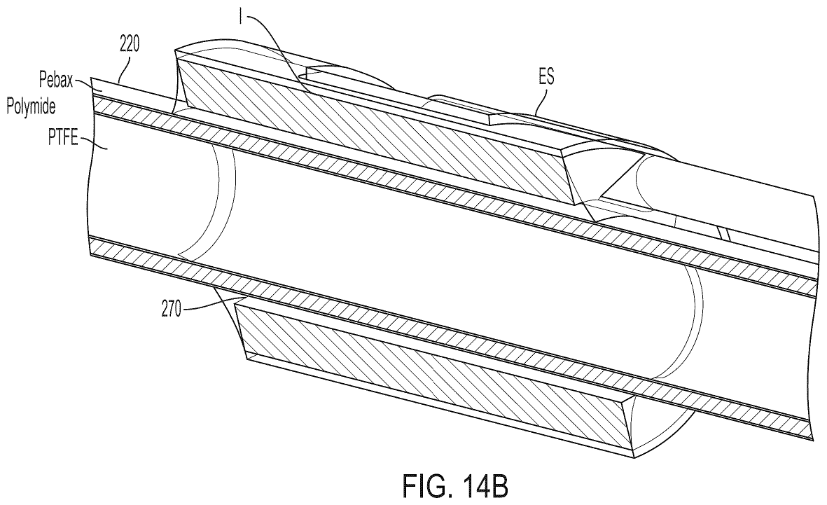

As shown in FIGS. 7B and 14B, the elongate member 220 to which the balloon 200 is sealed on the distal side comprises a polyimide core, which is lined on an inner surface with polytetrafluorethylene, commonly known as PTFE. The outer surface of the polyimide core is lined with 72D Pebax®. The electrode support members ES (proximal), ES′ (distal) may be stainless steel and are coated with an insulating material such as a polymer or a blend of polymers or other materials, including but not limited to an adhesive, polyimide or other high temperature resistant flowable non-conductive material.

Exemplary dimensions for the balloon 200 region may comprise the length of extension of the distal end of the outer member 230 into the inflatable section 210 a distance. An exemplary distance of extension of the outer member's distal end into the inflatable section is 0.794 mm, but other extension distances are within the scope of the present disclosure.

The balloon may be comprised of nylon or similar material. In some embodiments, the balloon material is uncoated, which may allow for more efficient transfer of energy from the pressure waves therethrough.

In addition, the presence of the tapering outer member 230 which adds stiffness to the device, the unbonded section 203 of the balloon 200, the tapering section 232 of the tapering outer member 230 which provides for a smaller crossing profile in a wrapped balloon configuration, all function to provide additional pushability and strength in the region of the outer member, and further works to prevent kinking of the wrapped balloon device during advancement through a patient's vasculature, both of which are highly advantageous.

As shown in FIGS. 6 and 9-11, a proximal marker band BP and a distal marker band BD may be provided within the balloon's inflatable portion 210 and disposed around the elongate member 220 at or near the transition from, respectively, the proximal and distal tapered sections 212, 214 into the substantially cylindrical section 216. In addition, a first proximal electrode support member ES is located along the elongate member 220 within the inflatable portion 210 at a position that may be closer to a proximal side of the balloon 200. In some embodiments, a single electrode support member ES may be provided as will be discussed further.

A second (distal) electrode support member ES′ may be located along the elongate member 220 within the inflatable portion 210 at a position that is spaced distally from the first (proximal) electrode support member ES and closer to the distal side of the balloon 200. The two electrode support members ES and ES′ are operatively and electrically connected by wire conductors W that are in operative electrical communication with the voltage pulse generator 10 as will be discussed further.

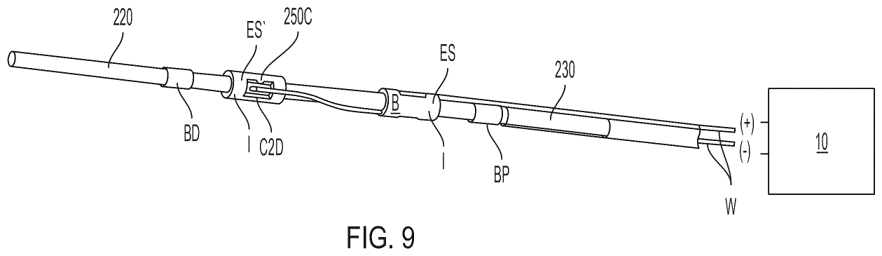

FIG. 9 illustrates a portion of the elongate member 220 and outer member 230 with the balloon 200 removed. Here, the first and second electrode support members ES, ES′ are shown in closer detail with the wire conductors W connected in a series connection. Each electrode support member ES, ES′ comprises a body B comprising a conductive material that is coated with an insulating material I and comprises at least one, preferably two, cutouts as will be discussed further. A tab or an arcuate region is defined on one of two longitudinal sides of each cutout and a wire conductor having an insulating covering with the exception of the distal-most end of the wire conductor which has no insulation As will be described further, a spark gap is formed between the exposed wire of the wire conductor and the tab or arcuate region of the cutouts of the electrode support members ES, ES′.

Each electrode support member may comprise two rotationally spaced cutouts with spark gaps which may be rotational spaced 180 degrees apart from each other, or may be spaced rotationally from each other at a different rotational spacing. As shown in FIG. 9, the spark gaps formed by the electrode support members ES and the spark gaps formed by the electrode support member ES′ may also be rotationally spaced from each other. For example, if the spark gaps of the electrode support members ES and ES′ are spaced 180 degrees apart around the respective electrode support member ES, ES′, then the two electrode support members ES and ES′ may be radially rotated to ensure that all spark gaps are rotationally spaced apart from each other to provide circumferential coverage. In some embodiments, two or more cutouts and the respective spark gaps may be longitudinally aligned. As best shown in FIGS. 6 and 9, the electrode support members ES and ES′ may be rotated relative to each other such that the respective cutouts and spark gaps are also rotationally spaced from each other around the elongate member 220. A preferred rotational spacing may comprise a 90 degree rotational spacing between spark gaps along the elongate member 220, though other rotational spacings are within the scope of the present invention.

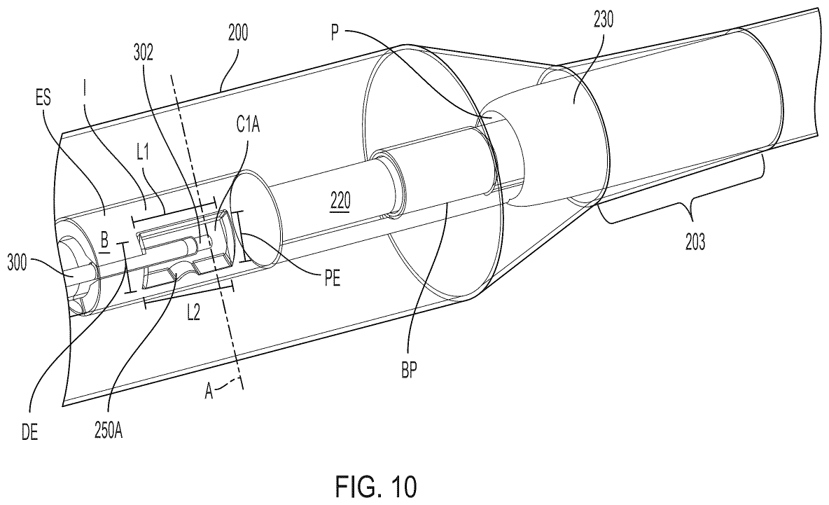

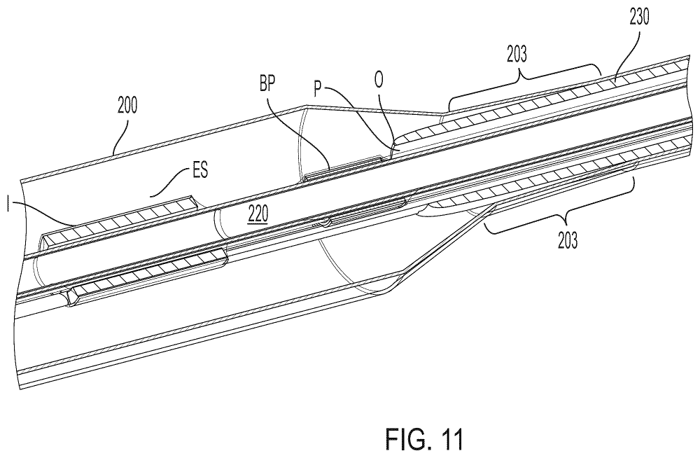

FIGS. 10 and 11 illustrate cutaway views of a proximal side of the balloon 200 and catheter structure, with a 1st (proximal) electrode support member ES shown with an associated wire conductor W. In addition, the wire conductors leading back to the positive and negative terminals of the voltage pulse generator occupy the fluid conveying pipe.

FIG. 10 also illustrates a proximal-most 1st electrode support member ES comprising a body B comprising a conductive material and defining a cutout CIA having two opposing longitudinal sides LI, L2 and opposing proximal and distal ends PE, DE. A tab or arcuate region 250 is formed or defined along one of the longitudinal sides LI. The surfaces of the electrode support body ES are covered in an insulating material I, with the exception of the tab or arcuate region 250, which comprises exposed conductive material.

As illustrated, a wire conductor 300 comprising insulation and having an exposed wire distal end region 302 that extends from a distal end a distance proximally. A proximal end region of the exposed wire region 302 is shown as located within the cutout, proximate to, and laterally or radially spaced from, the tab or arcuate region 250A. This configuration provides a pair of spaced-apart electrodes defining a spark gap between a lateral surface, and preferably not the distal end surface, of the exposed wire region 302 (comprising a first electrode in the illustrated spaced-apart pair of electrodes) and the tab or arcuate region 250 (comprising a second electrode in the spaced-apart pair of electrodes). In some embodiments, the distal end surface face of the wire conductor 300 may serve as an electrode in the above configuration.

The preferred embodiment comprises the lateral surface of the exposed wire conductor and that is located at a distal region of the wire conductor, serving as one of the electrodes in a spaced-apart electrode pair. The illustrated embodiment of FIG. 10 comprises the lateral surface of the exposed wire region 302 of the wire conductor as a first electrode, meaning that current flows to this electrode first, then across the spark gap to the second electrode of the spaced-apart pair of electrodes. As will be discussed, this current flow may be reversed in certain embodiments of the spaced-apart electrodes, wherein the metallic region (in the FIG. 10, the embodiment is a tab or arcuate region 250A) comprises the first electrode in the spaced-apart electrode pair. In this embodiment, the lateral surface of the wire conductor comprises the second electrode in the pair of spaced-apart electrodes and current flows to the first exemplary tab or arcuate region 250A, then across the spark gap to the second electrode comprising the lateral surface of the exposed wire region 302.

A preferred embodiment comprises the surface area of a first electrode and of a second electrode of a spaced-apart electrode pair to be substantially equivalent. In other embodiments, the surface area of a second electrode in a spaced-apart electrode pair may be larger than the surface area of a first electrode. Alternatively, the surface area of a first electrode in a spaced-apart electrode pair may be larger than the surface area of a second electrode.

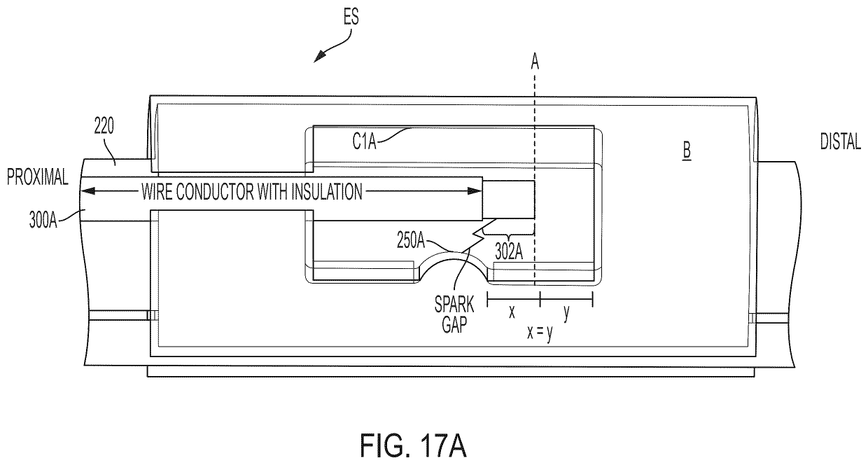

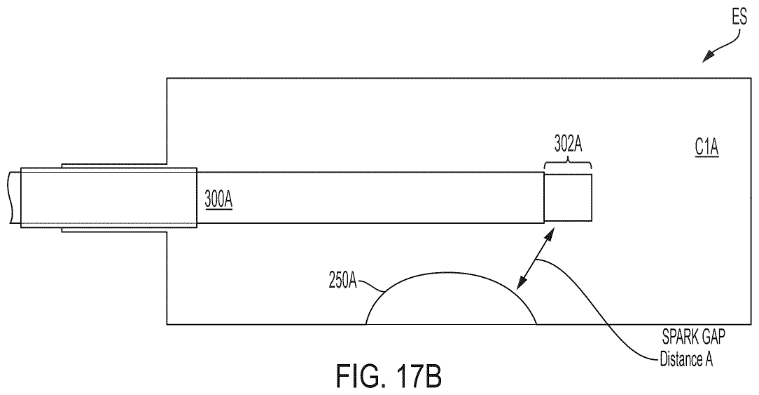

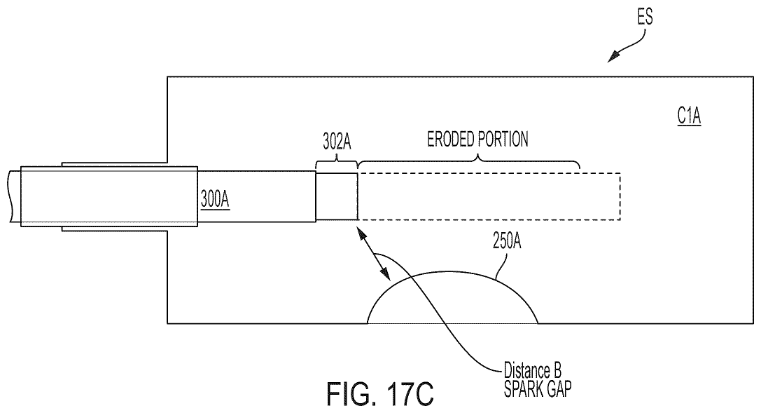

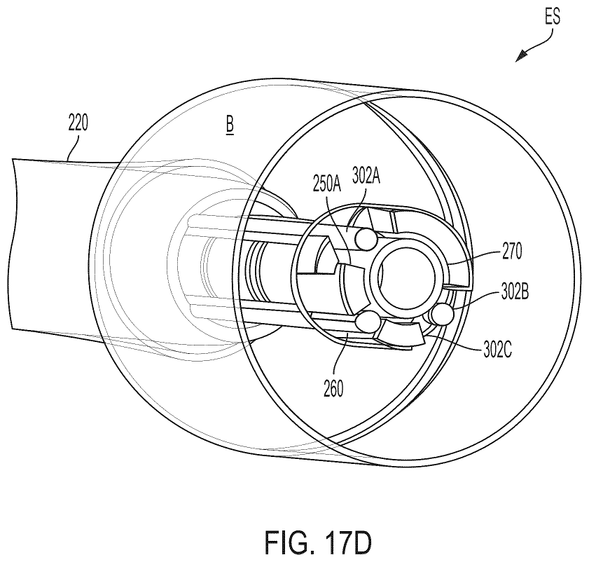

As will be discussed further, a preferred location for the exposed wire region 302 is to position the distal end approximately one half of the distance between the proximal end of the tab or arcuate region 250A and the proximal end PE of the cutout CIA. That location is shown by axis A in FIGS. 10, 17A-C, 18 and 19. In this configuration, the portion of the wire conductor directly overlaying the tab or arcuate region 250 remains insulated, with the lateral surface of the exposed wire region 302 being positioned beyond (in this case proximally beyond) the bounds of the tab or arcuate region 250. The spaced-apart electrodes of the embodiments described herein are preferably located along a common radial or circumferential plane.

Though the tab or arcuate region (2nd electrode) 250A is illustrated as positioned substantially mid-way along the length of the longitudinal side L1 of the cutout CIA, the tab or arcuate region 250A may be positioned more proximally or more distally as well. This changes the longitudinal position of the spaced-apart electrodes 250A, 302 as well as that of the defined spark gap between the spaced-apart electrodes 250A, 302, as well as effectively shifts the location and focus of the resultant pressure wave in the longitudinal direction to allow more effective coverage and/or interaction between adjacent generated pressure waves.

FIG. 11 illustrates a cutaway of the region illustrated in FIG. 10.

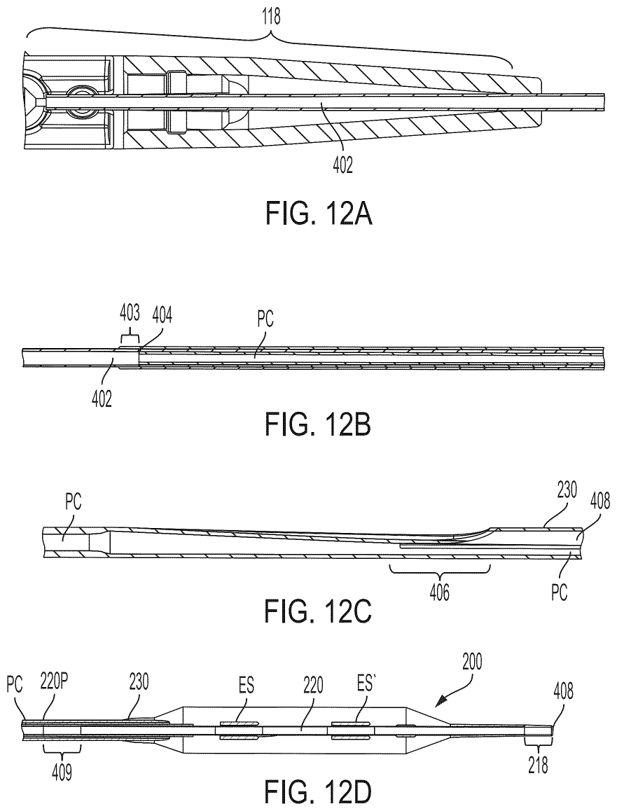

With continued reference to FIGS. 4-5 and 6-11, we now tum to FIGS. 12A-12D which illustrate features of an embodiment of an exemplary IVL system. FIGS. 12A-12D move successively distally along the exemplary system.

Beginning with FIG. 12A, just distal to the hub 118 as in FIG. 4, a hypotube 402 is provided which serves as a conduit for fluid infusion and removal and which is in fluid communication with the fluid reservoir and pump 114 as well as the interior of the inflatable portion 210 of the balloon 200. The hypotube 402 may be comprised of a metal and may be stainless steel. As shown in the combination of the structure shown in FIG. 12B, which is distal to the structure of FIG. 12A, an exemplary length of the hypotube 402 may be slightly longer than 1055 mm, though other lengths are within the scope of the present disclosure. FIG. 12B indicates a length of 1055 mm, however the hypotube 402 extends a slight distance further in the distal direction, along which it is overmolded by a 72D Pebax® material to form a bonded section 403 in order to, among other things, add bonding strength. The hypotube 402 is generally not coated on an outer or inner surface, but comprises a polymer near the distal end, e.g., Pebax® for aid in joining or bonding, and an adhesive on the proximal end region, also for aid in joining or bonding.

The hypotube 402 is shown as terminating distally at 404 in FIG. 12B. In some embodiments, the hypotube 402 may extend from the hub distally a distance of about 1080 mm. FIG. 12B continues a distance (e.g., and without limitation approximately 280 mm) distally from the bonded section 403 to comprise a polymide conduit PC for strain relief which is coated on its outer surface with the 72D Pebax®, which also comprises the bonded section 403.

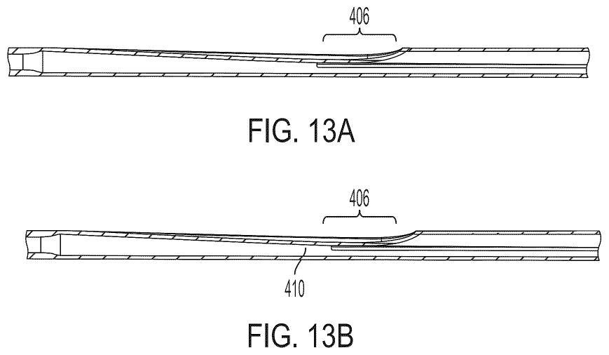

FIG. 12C illustrates the section comprising an RX port 406, which provides access for a guidewire or other interventional tools. The RX port 406 leads to a guidewire conduit 408 which may comprise a 63D Pebax® tube with an high density polyethylene (HDPE) such as Rezilok and having an inner surface that may be lined with 63D Pebax®. The guidewire conduit 408 extends distally through the polyimide conduit PC and the elongate member 230 and leads out of the system at the distal end of the distal tip 218. The RX port 406 is discussed further in FIGS. 13A and 13B.

A proximal end of the outer member 230 comprising 63D Pebax® that is lined with HDPE, e.g., Rezilok, is provided just distal to the RX port 406 as shown in FIG. 12C. The outer member 230 continues for a length or distance distally to terminate at a distal end that is located within the inflatable section 216 of the balloon as shown and described above, as well as illustrated in FIG. 12D. The elongate member 220 comprises a proximal end 220P that connects with the polyimide conduit PC described above and comprising a bonded, e.g., laser reflowed, section 409 to aid in bonding. The elongate member 220 extends through the outer member 230 and the interior of the balloon 200 to a point that is just proximal to the distal tip 218.

Turning now to FIGS. 13A and 13B, the RX port 406 is illustrated. As shown a hypotube 410 is provided, which may comprise a polymer, along a length of the polyimide tube on the proximal side of the RX port 406 for support. With supplemental reference to FIGS. 14A and 14B discussed below, known devices typically employ a support wire instead of the hypotube 410, but the inventors found that the polyimide tube with an outer polymer jacket comprising the elongate member 220 that transitions into the hypotube 410 solution provides greater and necessary stiffness and support in this critical region.

FIG. 14A illustrates a cross-sectional view of the tapering outer member 230, balloon 200, elongate member 220 with 1st and 2nd electrode support members ES, ES′. Electrode support members are in operative electrical communication with a voltage pulse generator 110 as discussed above. A bridge wire WT is provided for operative electrical connection between ES and ES′. The bridge wire is preferred to be made of tantalum rather than the known IVL devices which use copper wire throughout. Tantalum provides significantly enhanced durability as compared with copper. During testing, copper wire used as a “bridge wire” would slowly deteriorate or erode as the test voltage pulses and associated electrical arcs and current flow progressed. Ultimately, the copper bridge wire became susceptible to becoming displaced and interrupting the series connection between electrode support members ES and ES′. Thus, tantalum bridge wires were found to provide key durability characteristics, one of the features of the present disclosure that allows for a far greater number of electrical arcs (up to, and greater than, 500) to be produced than the known devices.

FIG. 14B is a closeup of a cross-section of an electrode support member of FIG. 14A. The elongate member 220 may comprise 3 layers: a core of polyimide which aids in resistance of heat generated by the electrodes during operation, an outer layer of Pebax and an inner layer of PTFE. Other polymers or blends of polymers may be used in the construction of the elongate member 220.

In some embodiments, an air or fluid gap 270 is provided between the electrode support member, for example ES and/or ES′, and the outer surface of the elongate member 220 to which the electrode support member ES and/or ES′ is adhered or operatively connected and at least partially surrounds. The air or fluid gap 270 functions to help to dissipate heat generated by the electrical arcs produced across the spark gap between the two spaced-apart electrodes 250, 302 described above, allowing the fluid within the inflated balloon 200 to flow through the air or fluid gap 270, and around and underneath a portion of the electrode support member ES and/or ES′ to remove heat from the structure. This is another key durability element of the present disclosure and contributes to the far greater number of electrical arcs, and at a greater frequency of electrical arcs, that are produced with embodiments of this disclosure compared with the known devices.

Further, the electrode support members, e.g., ES, ES′, are coated with an insulating material, with the exception of the bare metal electrode element, e.g., tab or arcuate region 250, and the wire conductors defining an exposed wire electrode element are also otherwise coated with an insulating material. As a result, the surface area of the spaced-apart electrodes in each case is relatively tightly controlled. This is in contrast to a known IVL system which provides concentric metallic electrodes with much more exposed metal surface area than is actually required and, as a result, produce much more undesirable gas as a by-product of generating electrical arcs. This is another key feature of the present disclosure that contributes to durability as well as improved variability when compared with known IVL systems.

The operational connection or adherence of the electrode support members ES and/or ES′ with the elongate member 220 in this embodiment is unique in that, as discussed above, the electrode support member ES and/or ES′ is coated or covered with an insulating material I. The insulating material I flows beneath portions of the electrode support member ES and or ES′ to form a connection or adherence between portions of the lower surface of the electrode support member ES and/or ES′ and the outer surface of the elongate member 220, while retaining the desired air or fluid gap 270.

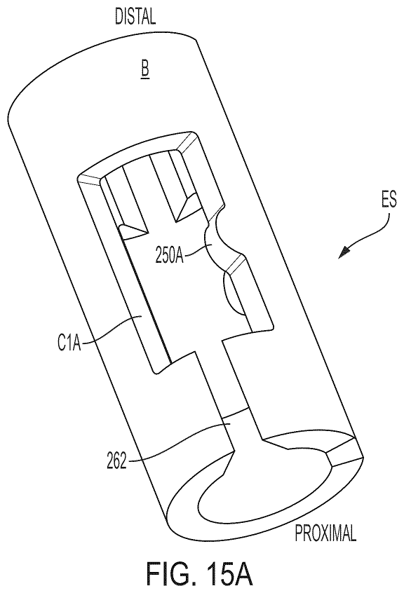

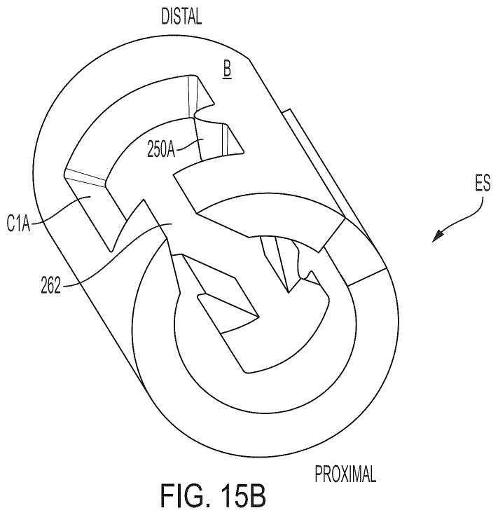

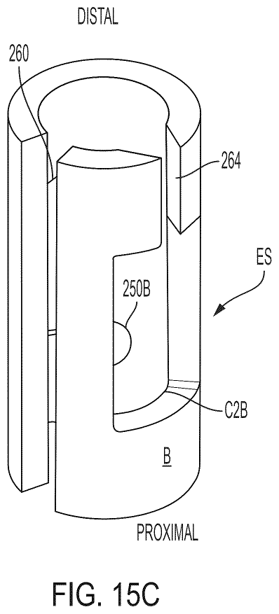

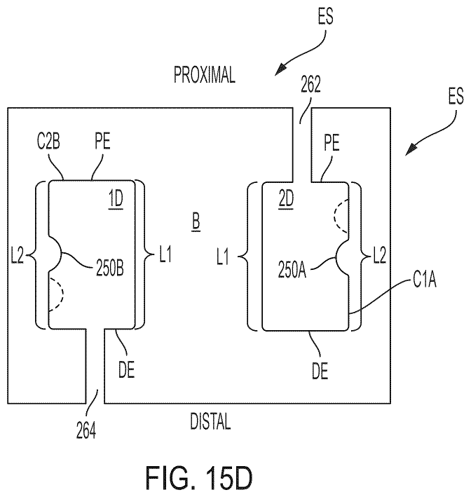

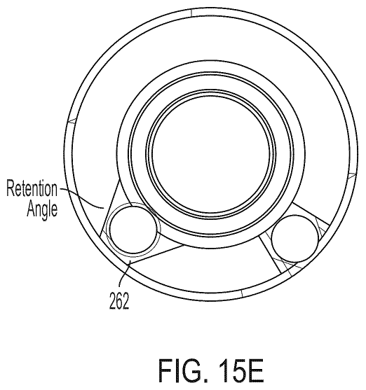

Turning now to FIGS. 15A-15E, one embodiment of an electrode support member ES comprising two electrodes is illustrated. The electrode support member ES may be used in the IVL system embodiments described above. The electrode support member ES comprises a body B which may be cylindrical and configured to at least partially surround the elongate member 220 as discussed above. In some embodiments the electrode support members discussed herein may not be fully circumferential as will be discussed further. The embodiment of FIGS. 15A-D comprises two radially spaced-apart cutouts, a first cutout CIA and a second cutout C2B. Each cutout CIA, C2B comprises opposing longitudinal sides LI, L2 and a proximal end PE and a distal end DE. The body B also comprises a longitudinally arranged slot or channel 260 that extends all the way along the body and configured to receive a portion of an insulated wire conductor. The first cutout CIA comprises a slot or channel 262 extending longitudinally away in a proximal direction from the proximal end PE of the first cutout CIA. The second cutout C2B comprises a slot or channel 264 that extends longitudinally away in a distal direction from the distal end DE of the second cutout C2B.

The electrode support member ES comprises a body formed of a conductive material which is covered with an insulating material I as described above. A region along one of the opposing longitudinal sides comprises exposed conductive material, with the insulating material covering removed. In the illustrated embodiment of ES, the exposed conductive material, e.g. metal, is provided along longitudinal sides L2 at 250A and 250B, respectively, for each the first and second cutouts CIA, C2B. The embodiment of FIGS. 15A-15E provides the exposed conductive material portion as individual arcuate regions 250A, 250B that extend radially into each cutout CIA and CIB, respectively. Each of the exemplary arcuate regions 250A, 250B defines one electrode of a spaced-apart electrode pair.

As best seen in FIG. 15C, the longitudinal slot or channel 260 running the entire length of the electrode support member body B is provided and is configured to receive insulated portion of one or more wire conductors. FIG. 15E illustrates a cross-sectional view through body B showing that the longitudinal slot or channel 262 may comprise angled sides, with a smaller opening at the outermost portion of the slot or channel 262. This angled retention structure may be used to retain wires in slots 260, 262 and/or 264.

The slots or channels 260, 262, 264 are provided to maintain a crossing profile of the electrode support members that is, at maximum, the outer diameter of the electrode support member body. In addition, the slots or channels 260, 262, 264 function to retain the wire conductors and associated electrode regions in the proper position within the subject electrode support member.

FIG. 15D provides an “unrolled” flattened view of the exemplary electrode support member body B. The electrode-defining arcuate regions 250A, 250B of exposed conductive material may be substantially centered along the longitudinal side L1 or L2 defining the arcuate regions 250A, 250B. Alternatively, as shown by the dashed lines, one or both of the arcuate regions 250A, 250B of exposed conductive material may be shifted away from the center of the longitudinal side. Thus, the arcuate regions 250A, 250B may be centered along the subject longitudinal side and/or shifted away from the center. In one embodiment, one of the arcuate regions, e.g., 250A may be offset longitudinally from the location of the other arcuate regions, e.g., 250B. This allows the locations of the arcuate regions 250A and 250B that are defined by a single electrode support member body B (and the resulting spaced-apart electrode, and defined spark gap locations) to be tuned and, in some embodiments, offset both radially and longitudinally from each other. This, in turn, allows for generation of pressure waves by a single electrode support body B with two radially spaced-apart electrode pairs, wherein the pressure waves produce mechanical forces that are not only offset radially from each other, but also offset longitudinally from each other.

The formation of the spaced-apart electrode pairs, and defined spark gaps, using exemplary embodiment ES are generally described above, and will be further discussed below.

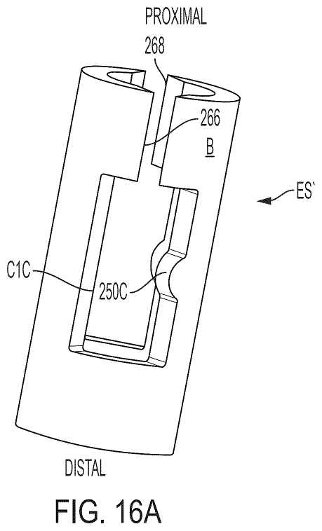

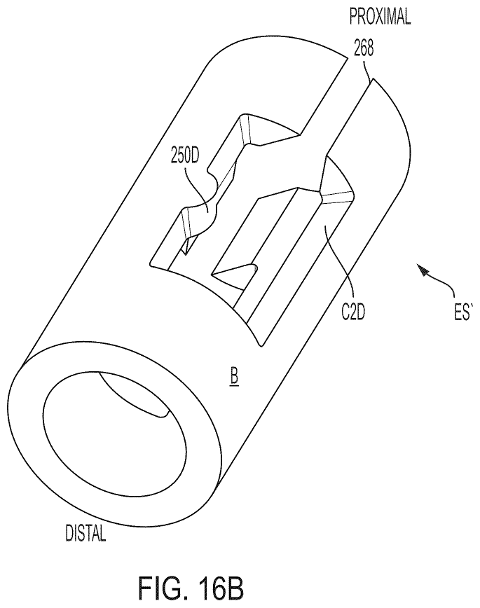

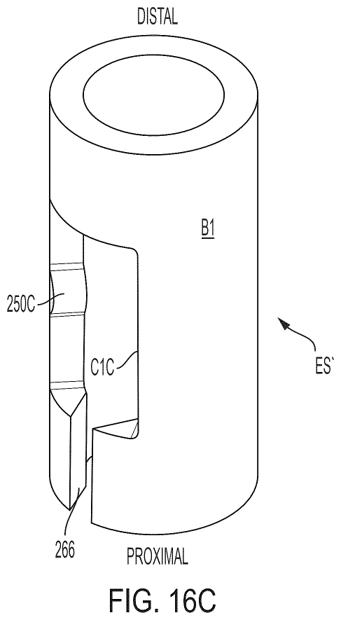

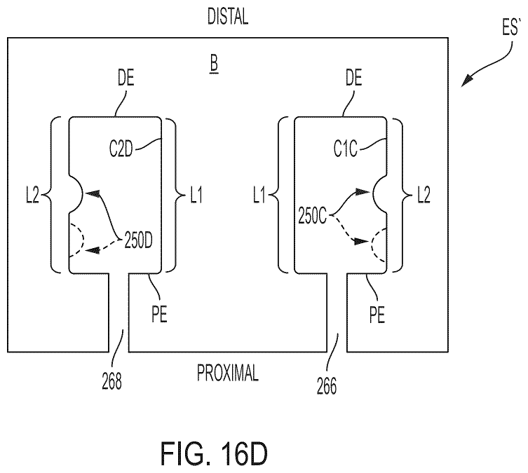

The exemplary electrode support member ES of FIGS. 15A-15E may comprise a proximal electrode support member such as shown in, e.g., FIG. 6 that is coupled with a more distally spaced-apart electrode support member ES′ which is discussed below in FIGS. 16A-16D. In addition, when two or more of the electrode support members ES may be electrically and operatively connected with each other and with a more distally spaced-apart electrode support assembly, one of the electrode support members ES may comprise a proximal-most electrode support member and the remaining electrode support members ES may comprise intermediate electrode support members located between a proximal and a distal electrode support member.

A distal electrode support member ES′ embodiment is illustrated in FIGS. 16A-16D. This embodiment may comprise a more distally located exemplary electrode support member ES′, such as shown in FIG. 6, when operatively combined with at least one more proximally-spaced apart electrode support member such as ES described above. More fundamentally and alternatively, this embodiment may be used alone, thus providing a single electrode support member ES′ with two radially spaced-apart electrode pairs when fully assembled. In an alternate embodiment, the single electrode support member ES′ may comprise a single pair of spaced-apart electrodes, wherein the spaced apart electrodes are in operative electrical communication with a first wire conductor that is in operative electrical communication with a first electrode of the electrode pair and a positive or high side terminal of a voltage pulse generator. A second wire conductor may be in operative electrical communication with a second electrode of the electrode pair and a ground or low side terminal of the voltage pulse generator.

The embodiment of FIGS. 16A-16D also comprises two radially spaced-apart cutouts, a first cutout CIC and a second cutout C2D. Each cutout CIC and C2D defines two opposing longitudinal sides L1, L2 and a proximal end PE and a distal end DE as well as a slot or channel 266, 268 extending in the proximal direction from the proximal end PE of each of the first and second cutouts CIC and C2D. As with the embodiment of FIGS. 15A-15E, exemplary arcuate regions of exposed conductive material 250C and 250D are defined along one of the longitudinal sides of cutouts CIC and C2D, respectively. As best seen in FIG. 16D, the regions of exposed conductive material, e.g., metal, 250C and 250D are each defined along longitudinal side L2 of the respective cutout CIC and C2D. Each arcuate region 250C and 250D forms and defines one electrode of a space-apart pair of electrodes. As with the embodiment of FIGS. 15A-15E, the location of one or both of the arcuate regions 250C and 250D (and the defined spark gap when the wire conductors are added, and the location of the pressure wave produced by the resultant spaced-apart electrodes) may be longitudinally shifted from the center of the defining longitudinal side(s) as shown with the dashed lines in FIG. 16D. This embodiment may not comprise the full length longitudinal slot 260 of the embodiment of FIGS. 15A-15E.

An exemplary spaced-apart electrode pair is illustrated in FIGS. 17A-17D. An exemplary electrode support member, which may be either of the embodiments discussed above in FIGS. 15A-16D, is provided. We will describe the illustrated embodiment as an electrode support member ES as described above in connection with FIGS. 14A-14D. A first cutout CIA is shown illustrated with a portion of an insulated wire conductor 300A located or received within the slot or channel 262 extending proximally away from the first cutout CIA. A distal-most region 302A of the wire conductor 300A is stripped of insulation, leaving an exposed distal-most region 302A of exposed conductive wire. Together with the exposed metal of the arcuate region 250A, the lateral surface, as opposed to the distal end face or surface, of the exposed distal most region 302a of the wire conductor 300A forms a spaced-apart electrode pair, with a spark gap defined therebetween.

The location of the exposed conductive wire region 302A is preferably positioned beyond the arcuate region as shown such that a distal end of the exposed wire region is halfway between the arcuate region and the distal end of the cutout CIA. This preferred dimensioning is illustrated by x and y, wherein x=y in FIG. 17A. Alternatively, the exposed wire region 302A at the distal end of the wire conductor may be positioned generally over the arcuate region to form an alternative embodiment of a spaced-apart electrode pair.

FIGS. 17B and 17C illustrate an exemplary starting and ending positions for the electrode comprising the exposed wire region 302A relative to the spaced-apart electrode comprising the exemplary arcuate region 250A. FIGS. 17B and C also illustrate a general direction of the current flow and electrical arc across the spark gap defined by the spaced-apart electrode pairs with distance A representing a starting spark gap length and distance B an ending spark gap length between the lateral face of the exposed wire conductor (exemplary first electrode) and the exposed conductive material of the arcuate region (exemplary second electrode). The current flow and resulting electrical arc will translate axially along with the translating exposed wire region 302A.

As the electrical arcing initiates and progresses between the spaced-apart electrodes 302A and 250A, the electrode comprising the exposed wire region 302A begins to erode and translate, effectively move axially (in the illustrative embodiment proximally) along the arcuate region 250A and engaging successively different (more proximal) regions of the arcuate region 250A in the electrical arcing process. The insulation initially covering the wire conductor 300A burns away, exposing successively more wire conductor and, as in FIG. 17C, with an exemplary ending position with a spark gap of distance B. As is now apparent, the spark gap changes relative locations, moving in this case in the proximal direction along a longitudinal axis of the wire conductor 300A. As the spark gap location changes, so does the effective direction of the current flow and resultant electrical arcs.

FIG. 17D illustrates a cross-sectional view through an exemplary electrode support member ES described in connection with FIGS. 15A-15E to show the relative positions and locations of exemplary first electrode comprising the exposed wire region 302A an exemplary second electrode comprising arcuate region 250A to define a first spaced-apart electrode pair. In addition, the relative positions and locations are shown for a second spaced-apart electrode pair, circumferentially or radially spaced away from the first spaced-apart electrode pair. The second spaced-apart electrode pair includes an exemplary second electrode comprising the exposed wire region 302B and an exemplary second electrode comprising arcuate region 250B. It is preferable that, in all embodiments described herein, the electrodes formed by the lateral face of an exemplary exposed conductive wire 302A, 302B and the respective and exemplary exposed metal region, illustrated as arcuate region 250A, B, be located at positions that are substantially the same relative to the outer surface of the elongate member 220. Stated differently, the spaced-apart electrodes formed by 302A, 250A and 302B, 250B may each be located a distance from the outer surface of the elongate member 220, wherein the distance from a spaced apart electrode 302A and 302B to the outer surface of the elongate member 220, and the distance from spaced-apart electrode 250A and 250B to the outer surface of the elongate member 220 are substantially equal. This arrangement provides an air or fluid gap 270 which is discussed further above. Moreover, with reference to FIGS. 15A-15E, at least part of the lateral surface of the exposed wire section of the first wire conductor may located between, and aligned with, the first and second longitudinal sides L1, L2 of the cutout such that the lateral surface of the exposed wire section is spaced apart from, and aligned with, the exposed metal region. Alternatively, the spaced-apart electrodes formed by 302A and 250A may each be located along the outer surface of the elongate member 220.

In all embodiments, the spaced-apart electrodes of the present disclosure are provided at substantially equal distances from a longitudinal axis through the IVL device's shaft.

In some embodiments, the spark gap represented by distances A and B may be of equivalent length. This is significant as it allows for use of a predictable and predetermined voltage magnitude to be provided which, in turn, produces a much more controlled pressure output from the produced pressure waves. Ultimately, the pressure output produced with a controlled, known spark gap length comprises more consistent, less variable forces than known IVL devices. This is understood to be critical in producing less strain on the balloon which, in tum, allows for a larger number of maximum voltage pulses, electrical arcs and produced pressure waves with a single catheter or system than is currently possible. For example, a known IVL coronary device has a maximum of 120 voltage pulses. The disclosed embodiment is demonstrated to effectively produce 300 voltage pulses per catheter in some embodiments and up to 500 pulses per catheter in some embodiments, with associated produced pressure waves, all within a very tight distribution and without significant decrease in the produced pressure outputs across the 300 voltage pulses. In some embodiments, the spark gap may be controlled such that it is within a predetermined range of length, with an exemplary minimum spark gap of 0.004″.

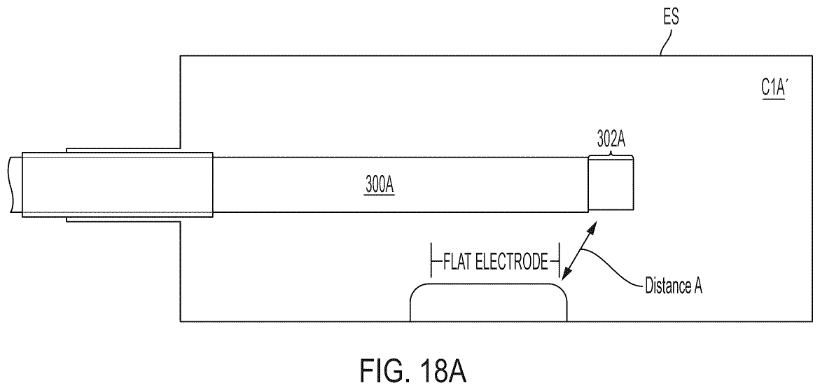

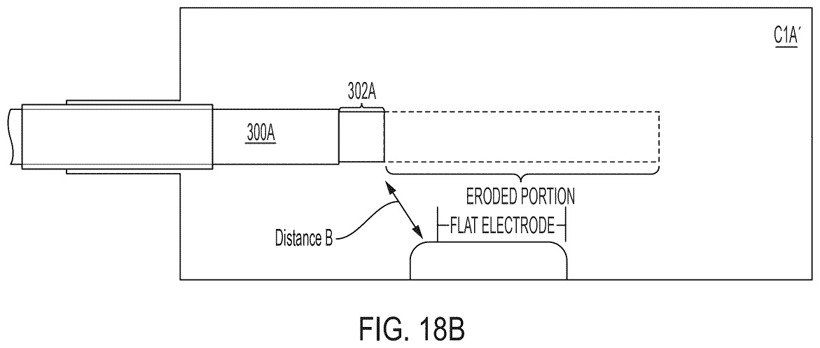

FIGS. 18A and 18B are similar to the embodiment of FIGS. 17A and 17B, wherein the arcuate region electrode is replaced with a cutout CIA′ comprising a raised flattened region that comprises exposed metal and functions as an electrode. This arrangement further ensures that, as the lateral surface of the exposed wire region 302A is involved in electrical arcs with the raised flattened region, the spark gap distance remains substantially the same across the executed series of voltage pulses and electrical arcs. Thus, distances A and B are substantially equal, and each spark gap therebetween is also substantially the same length as distances A and B.

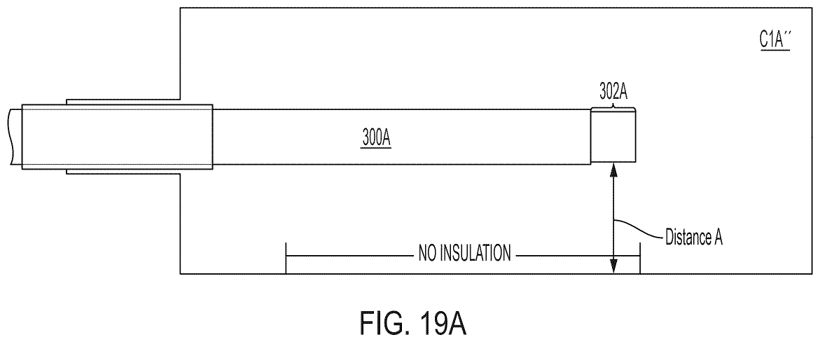

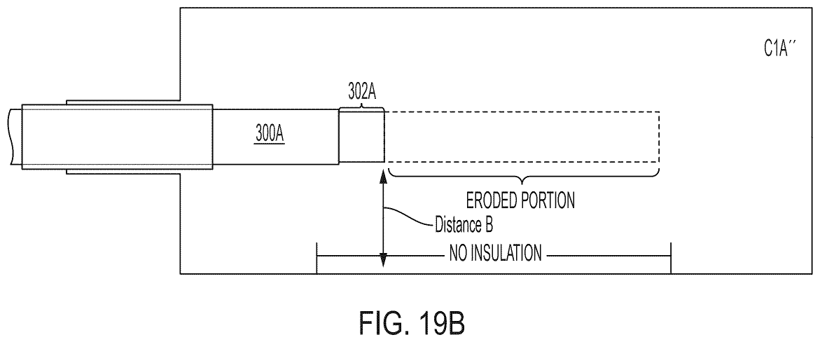

FIGS. 19A and 19B are alternative embodiments, similar in function with FIGS. 18A-18B except that the raised flattened region electrode is replaced with an inner surface of the cutout CIA″ of the electrode support member body B that is stripped of insulation for a length or distance, wherein the region of exposed conductive material functions as an electrode. This arrangement further ensures that, as the lateral surface of the exposed wire region 302 is involved in electrical arcs with the electrode comprising exposed conductive material, the spark gap distance remains substantially the same across the executed series of voltage pulses and electrical arcs. Thus, distances A and B are substantially equal, and each spark gap therebetween is also substantially the same length as distances A and B.

In some embodiments, the spark gap shaping and related distance as the erosion of the wire conductor's exposed wire lateral surface erodes may be tuned to the magnitude of the voltage pulses generated by the voltage pulse generator. In such embodiments, an initial series of voltage pulses at a predetermined magnitude, tuned to ensure that electrical arcs are produced between the spaced-apart electrodes at the known spark gap distance. In some embodiments, the spark gap distance may change at a known rate and within one or more of a plurality of series of voltage pulses and associated arcing as the electrical arcing process is executed. Thus, as the erosion process progresses, the lateral surface of the wire conductor's exposed wire is engaged in arcing and the exposed wire region, e.g. 302A, begins to translate and traverse over the electrode (exposed metal) surface of the electrode support member. During this shortening traversal, the spark gap distances may be substantially the same for a period of pulses/arcs and/or may change across the executed pulses/arcs. Changing spark gap distances may be correlated with the known spark gap distances relating to the relative positions of the lateral surface of the exposed wire of the wire conductor and the location range of produced electrical arc engagement along the exposed conductive material region of the electrode support member that comprises an electrode.

Thus, the controller may correlate the required, or desired, voltage pulse magnitude with the known spark gap distances, or distances ranges, for an initial series of voltage pulses and associated electrical arcs and further through to the maximum allowed number of voltage pulses and/or electrical arcs for a specific device. The known spark gap distances over time and produced arcs, allows the controller to modify the voltage magnitude as the pulse numbers progress (and the spark gap distances change) in order to ensure (1) that an electrical arc occurs; and/or (2) that the pressure output resulting from the controller-initiated voltage pulse at a predetermined magnitude is within a relatively tight and controlled window. In some embodiments, the controller may determine whether sufficient electrical energy was released by an energy storage element to produce an electrical arc.

The pairs of spaced-apart spark gaps may be arranged, and wired, in a variety of ways.

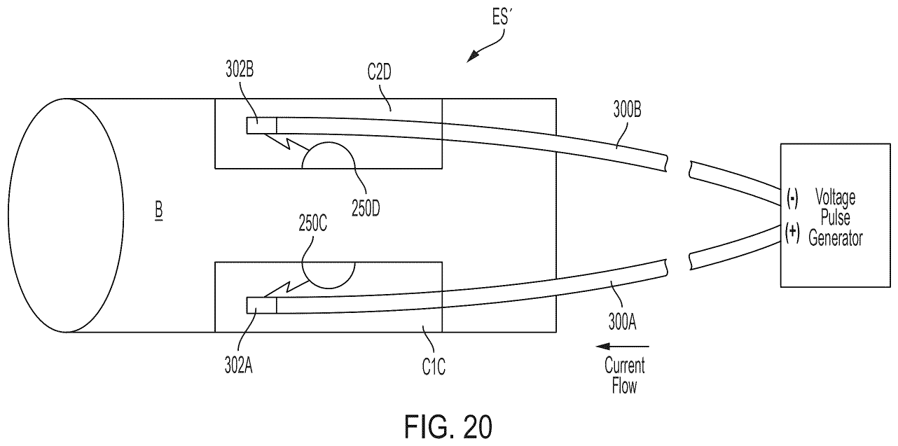

Perhaps the simplest arrangement involves the electrode support member ES′ discussed above in connection with FIGS. 16A-16D. As shown in FIG. 20, the electrode support member ES′ comprises a first cutout C1C with a first wire conductor 300A received within slot or channel 266 and with a first exposed wire 302A having a lateral face serving as a first electrode and positioned in a spaced-apart location relative to an exemplary exposed metal arcuate region 250C serving as a second electrode in a first spaced-apart electrode pair.

As discussed in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may be located between the first and second longitudinal sides of the cutout, and aligned with lateral surfaces of the first and second longitudinal sides of the cutout and with the exposed metal region. Alternatively, the two spaced-apart electrodes of an electrode pair may each be located along the outer surface of the elongate member 220.

The second cutout C2D also comprises an exposed metal arcuate region 250D which serves as a third electrode. Second cutout C2D also receives a second wire conductor 300B within slot or channel 268 and wherein a distal end of the second wire conductor 300B comprises an exposed wire region 302B with a lateral face thereof serving as a fourth electrode.

In operation, when the voltage generator initiates a voltage pulse of sufficient magnitude, current will flow through the first wire conductor 300A to the first electrode 302A and across a first spark gap from the first electrode 302A to the second electrode 250C within the first cutout CIC, creating an electrical arc and resultant pressure wave. Current will continue to flow through the conductive body B of the electrode support member ES′ until reaching the third electrode at exemplary arcuate region 250D of the second cutout C2D. Current will flow across a second spark gap from the third electrode to a fourth electrode which comprises the second wire conductor 300B and its distal region of exposed wire 302B, with the current then flowing along second wire conductor 300B back to a negative terminal of the voltage pulse generator. As the current flows across the second spark gap from the arcuate region 250D to the distal region of exposed wire 302B in the second cutout C2D, an electrical arc is generated with resultant pressure wave. The second wire conductor is in operative electrical communication with a negative or ground terminal of the voltage generator and the first wire conductor is in operative electrical communication with a positive or high side terminal of the voltage generator.

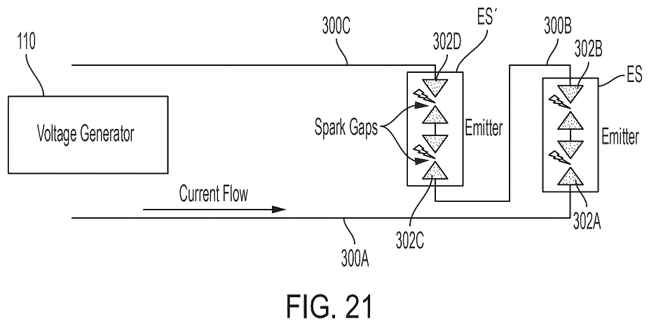

FIG. 21 illustrates two electrode support members connected in a series connection. With continued reference to FIGS. 15A-15E, the more proximally positioned first electrode support member may comprise electrode support member ES which, as discussed, comprises two radially spaced-apart cutouts CIA, CIB, each cutout CIA, CIB defining a pair of spaced-apart electrodes with a spark gap therebetween as described herein. The second (more distal) electrode support member may comprise ES′ as described above and, like ES, comprises two radially spaced-apart cutouts C2C, C2D, each cutout C2C, C2D defining a pair of spaced-apart electrodes with a spark gap therebetween.

With continued reference to FIGS. 15A-16D, the current flow in FIG. 21 initiates at the voltage generator producing a voltage pulse of sufficient magnitude. The resulting current flows distally along a first wire conductor 300A to reach a first electrode comprising the first wire conductor's distal region of exposed wire 302A. The lateral face of the exposed wire region 302A is in spaced-apart relation with a first cutout's CIA arcuate region 250A which serves as a second electrode in the spaced-apart electrode pair as described above. As described above in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may located between the first and second longitudinal sides of the cutout and aligned with the exposed metal region. Alternatively, the two spaced-apart electrodes of an electrode pair may each be located along the outer surface of the elongate member 220.

The current flowing across this first spark gap will create an electrical arc and associated pressure wave.

Continuing with reference to FIGS. 15A-16D and FIG. 21, current will continue to flow through the conductive material of the body B of the more proximally located electrical support member ES until reaching a third electrode comprising a second arcuate region 250B within a second cutout C2B that is radially spaced from the first cutout C2A. A second wire conductor 300B or bridge wire comprising tantalum provides a proximal and a distal end regions wherein both end regions comprise exposed tantalum wire, with the remaining tantalum wire covered in insulation. The proximal end of the exposed tantalum wire comprises a lateral face at 302B functions as the fourth electrode in this system and which is preferably in spaced-apart relation with the third electrode defined by the second arcuate region 250B.

As the current flows from the third electrode to the fourth electrode within the second cutout C2B, a second electrical arc is formed across the spark gap and a pressure wave results.

Next, the current flows along the second wire conductor 300B comprising the tantalum bridge wire to a more distally spaced and located electrode support member ES′. The distal exposed wire 302C of the tantalum bridge wire comprises a lateral face that serves as a fifth electrode in this system and which is located within a first cutout C1C of the electrode support member ES′. The lateral face of the fifth electrode is spaced apart from a sixth electrode comprising an arcuate region 250C of exposed metal of the first cutout C1C of the electrode support member ES′. As current flows from the fifth electrode across the defined spark gap to the sixth electrode, an electrical arc is generated and a pressure wave is produced.

Current continues to flow from the sixth electrode through the conductive body B of the electrode support member ES′ until reaching a seventh electrode comprising an arcuate region 250D of exposed metal within a second cutout C2D of the electrode support member ES′. An eighth electrode comprising a third wire conductor 300C having a distal end region of exposed wire 302D, wherein a lateral face of the exposed wire region 302D comprises the eighth electrode. The proximal end of the third wire conductor 300C is in operative electrical connection with a ground or low or negative terminal of the voltage generator. As current flows from the seventh electrode to the eighth electrode within the second cutout C2D of the electrode support member ES′, an electrical arc is generated across the spark gap and a pressure wave is produced. A portion of the third wire conductor 300C maybe received within slot or channel 260 of ES with a proximal end of the third wire conductor 300C placed in operative electrical connection with the voltage generator as discussed above.

Similar configurations may be produced with three or more electrode support members connected in series. For example, a proximal electrode support member ES may be serially connected via a tantalum bridge wire to a second more distal electrode support member ES which may, in tum, be connected via a second tantalum bridge wire to a distal most electrode support member ES′. The current will flow through the first proximal electrode support member ES as described above, then through the second more distal electrode support member ES in the same way, then through the distal electrode support member ES′ as described above.

It is also possible to combine two (or more) pairs of electrode support members connected in series, each pair of electrode support members functioning as described above, through use of a controller and/or multiplexer that selectively applies voltage to one pair of electrode support members, then selectively applying voltage to a second pair of electrode support members.

Moreover, in each of the wiring configurations for the embodiments of electrode support members discussed above, the current flow may be reversed within a given circuit by changing the polarity for a series of voltage pulses. In other embodiments, the polarity may be reversed for each voltage pulse. The result of such a polarity change is to change which side of the spaced-apart electrode pair functions as an anode and which side functions as a cathode. Functionally, this is may provide an advantage in terms of extending the number of resultant electrical arcs may be generated between the two spaced-apart electrodes, particularly if one of the spaced-apart electrodes wears or erodes more quickly than the other electrode in the spaced-apart pair of electrodes.

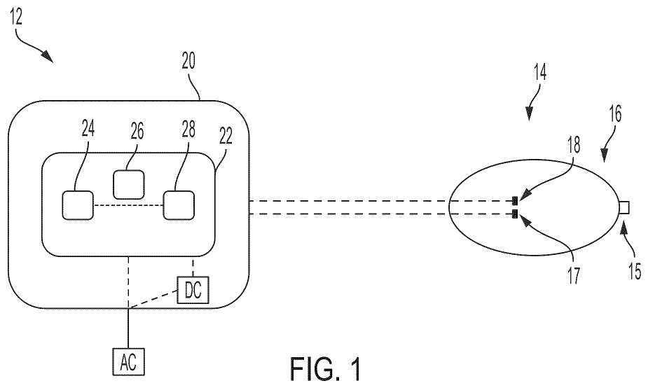

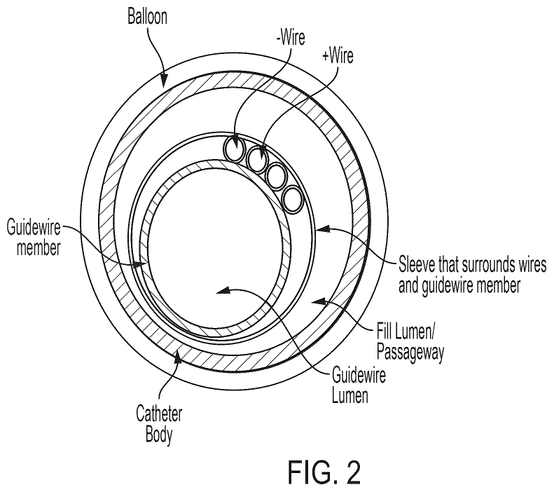

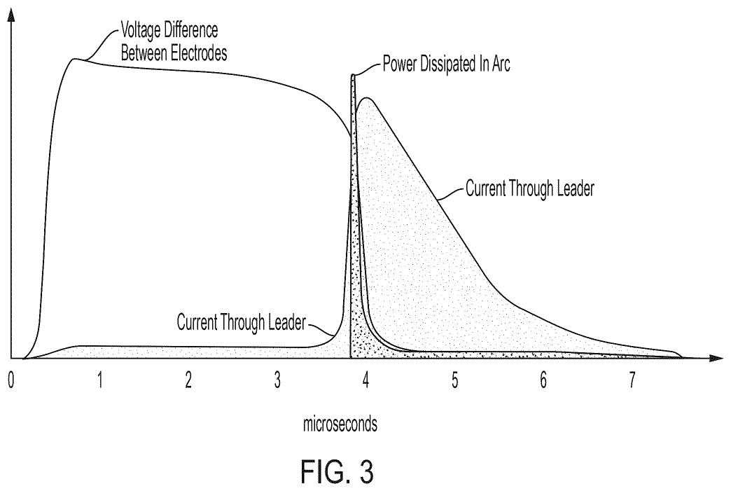

FIG. 22 illustrates a handle that may be in operative communication and connection with a console that may comprise a processor and an operatively connected memory such as described in connection with FIG. 1 for executing instructions and the voltage pulse generator 110 described above. In this embodiment, the handle comprises, or is in communication with, an EPROM (erasable read-only programmed memory) which may be in operative communication with a processor and/or a memory. The EPROM may provide therapy parameters, which may be specific for a particular model of IVL device, e.g., a balloon of specified length and/or with a specified number of electrode support members and/or spark gaps. In addition, the processor and/or memory may store therapy parameters for comparison of the progressing therapy monitored by the processor, against the stored therapy parameters. The processor may also generate connection logs, monitor number (and maximum number of allowed) pulses and may generate connection logs. FIG. 23 provides a general flow for data involving an EPROM with an IVL system of the present disclosure.

Having described certain key features of the exemplary IVL systems herein, we now turn to the functional results of those exemplary systems. A KNOWN IVL system operated in conformance with its instructions for use, and a TEST system conforming with the present disclosure were subjected to a comparative pressure output test.

The testing method and materials included comparison of TEST and KNOWN IVL devices, each including a catheter, a balloon, two radially spaced-apart pairs of electrodes within the balloon and a voltage pulse generator connected with the pairs of electrodes. The TEST balloon size included 2.5×12 mm and 4.0×20 mm. The KNOWN balloon sizes included 2.5×12 mm, 4.0×20 mm, 2.5×40 mm and 4.0×40 mm. Each tested IVL system comprised substantially similar spacing between longitudinally spaced and adjacent electrode pairs. The testing device included ONDA HNR-0500 (S/Ns 2149, Cal date 3 May 2023 and 2160, Cal date 5 May 2023) needle hydrophones without amplification. This hydrophone has an active diameter of 2.5 mm. The hydrophone was calibrated and traceable to Onda Corporation. The frequency response is flat from 0.5 MHz to 10 MHz within +/−6 dB with measurement uncertainty of 1.5 dB for frequencies 0.5-1 MHz and 1 dB for frequencies 1-10 MHz. The TEST and KNOWN devices were immersed in a water bath. A total of 1,440 voltage pulses were executed by the KNOWN system and the resulting pressure output measured, and a total of 13,320 voltage pulses were executed and the resulting pressure output measured for the TEST system using the testing method and materials.

The pressure output test method consisted of the following steps for each tested device:
1. Place the subject IVL device balloon in a water bath.
2. Position the hydrophone about 2.89 mm distal of the subject spark gap with an approximate 5 mm offset between the hydrophone and the catheter's longitudinal axis.
3. Generate a voltage pulse at a predetermined magnitude.
4. Move the catheter such that the hydrophone is positioned about 0.9375 mm proximally relative to the subject pair of electrodes' spark gap.
5. Repeat steps 1-4 until the hydrophone is positioned more than 2.89 mm proximal of the subject spark gap.
6. Repeat steps 1-5 at axial rotations of the catheter of 45, 90, 135 and 180 degrees.

### Comparative Testing and Select Features

### Pressure Output Variability

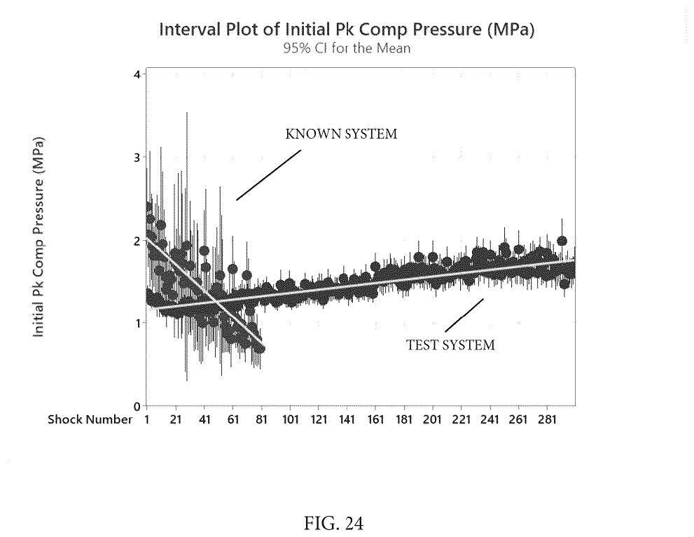

FIG. 24 provides a data plot of pressure output (initial peak pressures) for a KNOWN IVL system and a TEST IVL system that conforms with the present disclosure. As is immediately visually apparent, the KNOWN system's pressure output varies greatly. In contrast, the TEST system pressure output data is relatively tightly controlled, with relatively low variation compared with the KNOWN system's variation. A data summary is provided in Table 1:

TABLE 1

| | | | | Coefficient of |
|---|---|---|---|---|
| | Number | Mean | Standard Deviation | Variation (CV): |
| System | of Tests | (MPa) | (SD) | SD/Mean |
| KNOWN | 1,440 | 1.3945 | 0.5475 | 39.3% |
| TEST | 13,320 | 1.4468 | 0.3441 | 23.8% |

Thus, the coefficient of variation (“CV”), which is an indicator of variability within data sets is 39.3% for the KNOWN system's pressure output data and is 23.8% for the TEST system's pressure output data. Accordingly, it can be reasonably concluded that the TEST system's pressure output data is more controlled and substantially less variable than the KNOWN system's pressure output data. Using the key features in an IVL system will, therefore allow for a CV in a series of pressure output data that is less than 35%. More preferably, the CV in a series of pressure output data will be less than 30% and still more preferably, the CV in a series of pressure output data will be less than 25%. As noted supra, the IVL systems described herein provide for more durable (far more pulses/electrical arcs/pressure waves per catheter), more efficient (higher frequency of pulses/electrical arcs/pressure waves), and much more controlled pressure output from the produced pressure waves than the known IVL systems.

### Force Required to Traverse Vasculature

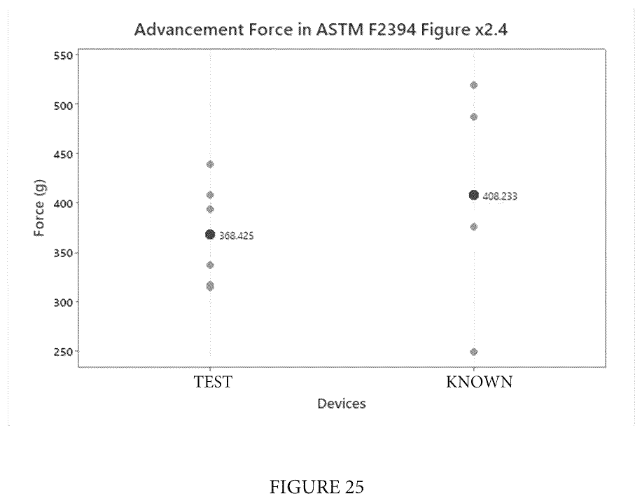

The TEST system and KNOWN system (Shockwave Medical C2 IVL catheter) were subjected to force standard tracking testing using the ASTM F2394 Tracking Fixture as shown in FIG. 25. The tested systems each comprises a total number of two emitters comprising spaced-apart electrodes. The tested systems were each translated through the illustrated standard fixture while tracking the force required to move the tested catheters from the beginning to the end of the fixture. The TEST and KNOWN systems were both two-emitter designs (two spaced-apart electrode pairs, wherein the spaced-apart electrode pairs are longitudinally spaced apart from each other), and the balloon sizes were both 3.0 mm×20 mm for both of the TEST and KNOWN systems. The ASTM F2394 Tracking Fixture was filled with water to lubricate. The TEST and KNOWN catheters were tracked over a wire through the model until the distal tip reached the end of the model. The peak force was the value that was recorded and observed in the plot as illustrated in FIG. 25. This comparative testing measures relative resistance through tortuous vasculature.

As shown, the mean peak force for the TEST system was 368.425 grams and for the KNOWN system, the mean peak force was 408.233 grams. This represents a 9.75% decrease in force required for the TEST system compared with the KNOWN system. The % difference between the two mean peak force values is 10.8%.

Accordingly, the mean peak force for embodiments of the TEST system through the ASTM F2394 Tracking Fixture is approximately 9.75% less than the mean peak force for the KNOWN system.

Certain embodiments of the TEST system may comprise a mean peak force through the ASTM F2394 Tracking Fixture that is at least 9.5% less than the mean peak force for the KNOWN system.

Certain embodiments of the TEST system may comprise a mean peak force through the ASTM F2394 Tracking Fixture that is at least 9% less than the mean peak force for the KNOWN system.

Certain embodiments of the TEST system may comprise a mean peak force through the ASTM F2394 Tracking Fixture that is at least 8% less than the mean peak force for the KNOWN system.

Certain embodiments of the TEST system may comprise a mean peak force through the ASTM F2394 Tracking Fixture that is at least 6% less than the mean peak force for the KNOWN system.

Certain embodiments of the TEST system may comprise a mean peak force through the ASTM F2394 Tracking Fixture that is at least 5% less than the mean peak force for the KNOWN system.

Accordingly, the TEST system traversed the test fixture from a beginning to an ending of the fixture with a peak force less than 405 grams.

Further, the TEST system traversed the test fixture from a beginning to an ending of the fixture with a peak force less than 400 grams.

The TEST system also traversed the test fixture from a beginning to an ending of the fixture with a peak force less than 375 grams.

The TEST system also traversed the test fixture from a beginning to an ending of the fixture with a peak force less than 370 grams.

As a result, the peak advancement force for the TEST system may be within a range of about 400 grams to about 405 grams.

The peak advancement force for the TEST system may also be within a range of about 375 grams to about 405 grams.

The measured peak advancement force is for the TEST system may also be within a range of about 370 grams to about 405 grams.

In addition, Table 2 below provides a partial summary listing of functional improvements and enhancements provided by embodiments of the present disclosure relative to a KNOWN system, with a partial listing of the disclosed features leading to the improvements and enhancements.

| | | | Presently |
|---|---|---|---|
| | | Presently | Disclosed |
| Functional | | Disclosed | Features Enabling |
| Result | KNOWN System | Embodiments | the Functional |
| Crossing | Outer diameter of | Outer diameter | 1. Electrodes not |
| Profile | 0.044″-0.047″ | (“OD”) about | fully |
| | | 0.044″ or less for | circumferential; |
| | | 2.5 mm diameter | wires m same |
| | | balloon. OD | circumferential |
| | | about 0.045′ or | plane as the body |
| | | less for 3.0 mm | of the electrode |
| | | and 3.5 mm | support member. |
| | | diameter | 2. Tapering |
| | | balloons. | member in the |
| | | | proximal inflation |
| | | | region of the |
| | | | balloon. |
| Deliverability | | | 1. Smaller |
| | | | crossing profile |
| | | | in the folded |
| | | | balloon region; |
| | | | less push force |
| | | | required. |
| | | | 2. Smaller distal |
| | | | outer diameter at |
| | | | distal tip than at |
| | | | the proximal end |
| | | | of the sealed |
| | | | balloon. |
| | | | 3. Increased |
| | | | flexibility in at |
| | | | least the balloon |
| | | | region compared |
| | | | with KNOWN - 1 |
| | | | less layer of |
| | | | material than |
| | | | KNOWN. |
| | | | 4. Improved axial |
| | | | push force |
| | | | without kinking |
| | | | due to presence of |
| | | | tapering outer |
| | | | member and |
| | | | stiffening layer |
| | | | in region of the |
| | | | balloon. |
| Efficiency/ | 1 pulse/second | 1-5 pulses/second | 1. Control of |
| Frequency of | | with 2 pulses/ | spark gap lengths. |
| Pulses | | second preferred | 2. Minimal spark |
| | | | gap shorting. |
| | | | 3. Tantalum |
| | | | bridge wire. |
| | | | 4. Insulation |
| | | | covering |
| | | | electrode support |
| | | | bodies, resulting |
| | | | in less gas |
| | | | produced during |
| | | | arcing. |
| Variability of | Greater than | Less than | 1. Control of |
| Pressure | 39% CV | 24% CV | spark gap lengths. |
| Output | | | 2. Minimal spark |
| | | | gap shorting. |
| | | | 3. Tantalum |
| | | | bridge wire. |
| | | | 4. Changing |
| | | | voltage to |
| | | | accommodate |
| | | | pressure over |
| | | | number of pulses. |
| Durability | Maximum 120 | Up to 500 pulses | 1. Control of |
| (maximum | pulses per | per catheter for | spark gap lengths. |
| number of | catheter for | coronary and | 2. Minimal spark |
| pulses per | coronary devices. | peripheral | gap shorting. |
| catheter). | Maximum 300 | devices. | 3. Tantalum |
| | pulses per | | bridge Wire. |
| | catheter for | | 4. Insulation |
| | peripheral | | covering |
| | devices. | | electrode support |
| | | | bodies, resulting |
| | | | in less gas |
| | | | produced during |
| | | | arcing. |
| | | | 5. Less variable |
| | | | pressure output |
| | | | than known |
| | | | systems. |

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. An IVL system comprising:
a pulse generator 110 comprising positive and negative terminals and an interconnect;
a cable with a proximal end and having an interconnect at the proximal end that is configured to interconnect 120 with the interconnect of the pulse generator 110;
an actuating handle located along the cable;
an elongate member 220 having an outer surface;
a fluid-inflatable balloon 200 located at or near a distal end of the elongate member 220,
wherein one or more pairs of spaced-apart electrodes are disposed within the fluid-inflatable balloon 200,
wherein a first one of the one or more pairs of spaced-apart electrodes comprises: a first electrode 302A comprising a first wire conductor 300A surrounded by an insulator and operatively connected with the positive terminal of the pulse generator 110 at a proximal end and comprising a distal end, wherein the first wire conductor 300A comprises an exposed wire section 302A defining the first electrode 302A that extends a length proximally from the distal end;
a second electrode spaced apart from the first electrode and formed from a first electrode support member ES having a body B with a length that is affixed with, and at least partially surrounds, an outer surface of the elongate member 220; and
a slot 260 defined along the length of the body B,
wherein the first electrode support member body ES comprises a conductive material that is covered with an insulating material I and defines a first cutout C1A with an arcuate region defining the second electrode 250A located along one longitudinal side of the first cutout and extending toward an opposing longitudinal side of the first cutout, wherein a lateral surface of the arcuate region defining the second electrode 250A is stripped of the insulating material, and
wherein the exposed wire section defining the first electrode 302A extending from the distal end of the first wire conductor is positioned within the first cutout C1A, such that a lateral surface of the exposed wire section defining the first electrode 302A is located between, and aligned with, the longitudinal sides L1, L2 of the first cutout C1A, and spaced radially apart from the lateral surface of the arcuate region defining the first electrode 302A of the first cutout C1A, to define a spark gap between the lateral surface of the exposed wire defining the first electrode 302A and the lateral surface of the arcuate region defining the second electrode 250A.

51. The IVL system of claim 50, wherein the first electrode is spaced a distance from the outer surface of the elongate member, and the second electrode is spaced a distance from the outer surface of the elongate member, wherein the distances from the outer surface of the elongate member to each of the first and second electrodes are substantially equal.

52. The IVL system of claim 50, wherein at least part of the lateral surface of the exposed wire section of the first wire conductor is located between the first and second longitudinal sides of the cutout and aligned with the lateral surface of the arcuate region.

53. The IVL system of claim 50, wherein the first cutout comprises a first channel on a proximal end of the first cutout that extends away from the first cutout in a proximal direction, and wherein a portion of the distal end of an insulated portion of the first wire conductor is received within the first channel.

54. The IVL system of claim 50, wherein the distal end of the first wire conductor is initially located at a position that is one-half of the distance between a distal side of the arcuate region and the distal end of the first cutout.

55. The IVL system of claim 50, wherein the exposed wire section of the first wire conductor is initially located distal of the arcuate region.

56. The IVL system of claim 50, wherein a portion of the exposed wire section of the first wire conductor is initially located alongside a portion of the arcuate region.

57. The IVL system of claim 50, wherein the distal end of the first wire conductor is located at a position that is one-half of the distance between a center of the arcuate region and the distal end of the first cutout.

58. The IVL system of claim 50, further comprising a ground wire conductor in operative electrical communication with the second electrode and with the negative terminal of the pulse generator.

59. The IVL system of claim 50, further comprising a second one of the one or more pairs of spaced-apart electrodes, comprising:
a third electrode defined by the first electrode support that comprises a second cutout that is circumferentially spaced apart from the first cutout, wherein the second cutout defines an arcuate region with a lateral surface that is stripped of the insulating material and located along one longitudinal side of the second cutout and extending toward an opposing longitudinal side of the second cutout, wherein the third electrode is in operative electrical communication with the first one of the one or more pairs of spaced-apart electrodes; and
a fourth electrode spaced apart from the third electrode and comprising a second wire conductor surrounded by an insulator wherein the second wire conductor comprises an exposed wire section, wherein the exposed wire section is located between, and aligned with, the longitudinal sides of the second cutout, and spaced radially apart from the lateral surface of the arcuate region of the second cutout to define a spark gap between the lateral surface of the exposed wire section of the second wire conductor and the arcuate region of the second cutout.

60. The IVL system of claim 59, wherein the third electrode is located a distance from the outer surface of the elongate member, wherein the fourth electrode is located a distance from the outer surface of the elongate member, wherein the distances of the location of the third electrode and the fourth electrode from the outer surface of the elongate member are substantially equal.

61. The IVL system of claim 59, wherein the second cutout further comprises a second channel on the proximal end of the second cutout and wherein a portion of the proximal end of an insulated portion of the second wire conductor is received within the second channel, and wherein the second channel extends proximally away from the second cutout.

62. The IVL system of claim 59, wherein the distances of the first, second, third and fourth electrodes from the outer surface of the elongate member are substantially equal.

63. The IVL system of claim 59, wherein at least part of the lateral surface of the exposed wire section of the first wire conductor is located between the first and second longitudinal sides of the cutout and aligned with the lateral surface of the arcuate region.

64. The IVL system of claim 60, wherein a distal end of the second wire conductor is located at a position that is one-half of the distance between a distal side of the arcuate region of the second cutout and the distal end of the second cutout.

65. The IVL system of claim 60, wherein the exposed wire section of the second wire conductor is located distal of the arcuate region of the second cutout.

66. The IVL system of claim 60, wherein a portion of the exposed wire section of the second wire conductor is located alongside a portion of the arcuate region of the second cutout.

67. The IVL system of claim 60, wherein a proximal end of the first wire conductor is located at a position that is one-half of the distance between a center of the arcuate region and a distal end of the second cutout.

68. An intravascular lithotripsy (IVL) system comprising:
a pulse generator 110 comprising positive and negative terminals and an interconnect;
a cable with a proximal end and having an interconnect 120 at the proximal end that is configured to interconnect with the interconnect of the pulse generator 110;
an actuating handle located along the cable;
an elongate member 220 having an outer surface;
a fluid-inflatable balloon 200 located at or near a distal end of the elongate member 220, wherein one or more pairs of spaced-apart electrodes are disposed within the fluid-inflatable balloon,
wherein a first one of the one or more pairs of spaced-apart electrodes comprises: a first electrode 302A comprising a first wire conductor 300A surrounded by an insulator and operatively connected with the positive terminal of the pulse generator 110 at a proximal end and comprising a distal end, wherein the first wire conductor 300A comprises an exposed wire section comprising the first electrode 302A that extends a length proximally from the distal end; and
a second electrode 250A spaced apart from the first electrode 302A and comprising a first electrode support member ES having a body B that is affixed with, and at least partially surrounds, an outer surface of the elongate member,
wherein the first electrode support member ES body B comprises a length, a slot 260 along the length of the first electrode support member body, and a conductive material that is covered with an insulating material I and defines a first cutout C1A with an exposed metal region defining a second electrode 250A located along a first longitudinal side of the first cutout,
wherein a lateral surface of the exposed metal region is stripped of the insulating material I, and
wherein the exposed wire section extending from the distal end of the first wire conductor and defining the first electrode 302A is positioned within the first cutout C1A, such that a lateral surface of the exposed wire section defining the first electrode 302A is located between, and aligned with, the longitudinal sides L1, L2 of the first cutout C1A, and spaced radially apart from the lateral surface of the exposed metal region of the first cutout C1A defining the second electrode 250A to define a spark gap between the lateral surface of the exposed wire of the first electrode 302A and the lateral surface of the exposed metal region of the second electrode 250A.

69. An intravascular lithotripsy device comprising at least one pair of spaced-apart electrodes in operative electrical connection with a voltage pulse generator, comprising:
a first electrode 302A comprising:
a first wire conductor 300A comprising a proximal end and a distal end, wherein the first wire conductor 300A is surrounded by an insulator I and operatively connected with a positive terminal of the pulse generator 110 at the proximal end of the first wire conductor 300A, wherein the first wire conductor 300A comprises an exposed wire section comprising a first electrode 302 A having a lateral surface that extends a length proximally from the distal end; and
a second electrode 250C spaced apart from the first electrode 302A to form a spark gap therebetween and comprising:
an electrode support member ES′ having a body B,
wherein the first electrode support member body B comprises a conductive material that is covered with an insulating material I and defines a first cutout C1C with an exposed metal region located along a first longitudinal side L1 of the first cutout C1C, wherein the exposed metal region comprises a lateral surface that faces toward a second, opposing longitudinal side L2 of the cutout C1C, and a channel 266 leading away from the first cutout C1C in a proximal direction,
wherein a portion of the first wire conductor 300A is received within the channel 262, and
wherein the exposed wire section of the first electrode 302A is located between, and aligned with, the longitudinal sides L1, L2 of the first cutout C1C, and spaced radially apart from the lateral surface of the exposed metal region defining the second electrode 302A to form a spark gap between the lateral surface of the first electrode 302A and the second electrode 250C of the first cutout C1C.
